SEQUENCE LISTING (268 sequences)

SEQ ID NO: 1 - DNA, 200 bp, Tetranychus urticae
accaataaacatttccttgtggtgaaactcggcagcccatataaaaaggtatcgcagttactctaacattttctgtggtttctttatggacatcagataactctaaccaagggtgatttttttcttgccatgcttttaaagtatctttagggacaaaaggagggtctcgttcatgatcacgagccaaaaatttctcgaat

SEQ ID NO: 2 - DNA, 217 bp, Tetranychus urticae
tactgttattattgttaggttgattggcggggttaactgagaaagaattaaatgctaatgaattcctggatccaatcggtgggcaacttcgaggggcaatccattgattggtataatcaaaactgttcaatcccttggtattaaatggattactaccaataccactgatagtatgagcgagattacttgaataaagactaccattattattggtacc

SEQ ID NO: 3 - DNA, 470 bp, Tetranychus urticae
tacttggtgcacttgtaacaatacggtgtcctcgttacagtataatttaaccagttcatcgtccattagcagggtaattcctttcaaacatcttttatagaaatgtcttatattcttagcttccaatttgtactttttctgaatagataacgctaaaccgaccagtgttggagggacaatgtgcaatggatggaatatttcttcgttttcctgtttagcatatagcaacaatcgatctggaggataaactttgtcccttttaggtggaggctgagttacaaccatttccagattggcggaaataggatcggcacttcgttttcttttgtcaattattcctggtgaagctgttccagaggatcctggtggagttatgatgggtaaatgggtaggtgaatcatatcctgcagtgttcaagtctccagttgaatgttggcttgagtaaaagctcatatcgcctgtggttatct

SEQ ID NO: 4 - DNA, 239 bp, Tetranychus urticae
ccactgttggtgtaagttgtgaattgcttctagcagctttgcgttcagattttggcgatggtagtgggaatttggttgaaactagagctaaagaggctgcagcatgttgagcttttcttattgatggagcattagcacgatactcttctttatcgcccaatttcaggacaactgaaaagatctttgaatgaggaactccagattcttcaatcagctcatatcgacaagtgattgaattg

SEQ ID NO: 5 - DNA, 184 bp, Tetranychus urticae
acaagtgattgaattgaatcgacaaagctcattaaccaagcacattggacttttatctttatgtttgttgactatattttcagattggttgctatcattggatgcggttgtcatttcagcatcaatttctgtacctttttgatctgaagatactgacaaagaggtgttttggttcacattggaa

SEQ ID NO: 6 - DNA, 177 bp, Tetranychus urticae
tgagatgagtatttacaggggaacagcaaattcatagcaagtttctccatttgaagattacgtatgttatttgaaataggaatacgacaagtggggcaattagttaattgtttgcgacacgcagagcacaaaagatgtccattttgacactgaagaataggaggaagaacgtaatca

SEQ ID NO: 7 - DNA, 198 bp, Tetranychus urticae
taaacaggagaaatggtgacatttatacccagattcgcaccatctgcaaaaagattcgcaatacttgtgtcaaaaactaagcaatcagatagattaatagcactctgaacaccttcatcgatagaatgtggagttgcttcccatgttaatcttcttttttgtccgtttaattcgagacgataaataaatttttctgct

SEQ ID NO: 8 - DNA, 171 bp, Tetranychus urticae
tgattgaattcactttttcgcacatcatagctccaagtgatttaaccttgattgtacttccataaaggtcgaagaaaggatagaaactttgagtttgatcaatatgcatcaaagtaattggaggttgacggtttttggtcataataacttggccagattcagttattttaa

SEQ ID NO: 9 - DNA, 427 bp, Tetranychus urticae
ctaaaaatctaattgcagtggtagatccataaatatcgagtaaagcccatatcgtttttgaaacatcaacttcagttaaaaatactcctttgtcatcaccgttgattgagtaatgagctttaccagattcatcgacccagtacgataaaacattttcgacaacaccataacgatcacttaatgctttggcccagaatccagatttagaggttaactcagggcaagcatatttgggtagttgacccttcaatttgattggatctgttgtagtgaatccaaaacgaagagcaccattccaattggttgaagaggtcataaatttaattgaaacatgttcgccaactttaattggacgagatgagaaaacaattccttcacaaaaactttccaatcgttcagcaatggctccattgccagataaacgaat

SEQ ID NO: 10 - DNA, 188 bp, Tetranychus urticae
taagcataataagttctgataacatccaaaattcgtgctcgacaaagaggacatcgaccaccattttgccctttccattgtttaactgcacaatcgtaacacatacacatatgaccacaagtgtagaggacacaattaatgggcgcctcaaaacaaactgtgcattccgactcaacattcaaagatcc

SEQ ID NO: 11 - DNA, 217 bp, Tetranychus urticae
tggattgttgatggttagactcgcgaggtgatgaagactctcgacctggctcaggactgcccttgccttcatggccacttcgactgggtgaccgggaagaaggtaaattaagaccgggtaacatgtgcggaagtccaaggtaacctgctccattgactagagaggcagcggcaagacgagcaaatacttcaggatcagttgcagccgcagcagccgc

SEQ ID NO: 12 - DNA, 192 bp, Tetranychus urticae
attgggataaaagtgaatttgtaattgattgcattcgctgtaagggtgatgctgaggctgataaatcttcagcaccaaccgatccactattgttgttggcaacactgttgctattggcactgccattgttacttgtgtttgcattggttgctgttggagttgataaattagaggtggaagaagatgatgacc

SEQ ID NO: 13 - DNA, 190 bp, Tetranychus urticae
agaaacattttcattctaatgaaaggttcccgacccttttgggtcaacatgtgccatggtttgggacgagcaagcaaatcagaaactgaaccctgggaaagtcctaaaactgattcaccgaaaagtcgttgactaattgaatactgacttagctgttctttaaccttcttgacgatgtcttcagtattta

SEQ ID NO: 14 - DNA, 381 bp, Tetranychus urticae
aatttgtccacattttgtcgtaaagcttcttgcagcttttgaagtgacatttcaagagatcgatgttgactttcaagggaaactaaatttgtaccgcttccaataataccaagagtagtgttgtttaatgaagacaacggatgattgccagttgatcggctattgctatcattacctgtaccagaaccagcagtgttgccagttaaaatgatgccctctaaaggtgcgcttccatgatggaaacccagtaaacctggattggtcaacaagtctccatgggcagagatgatgtttgaggttaatgatgcggtggtttttaaaggtccagaggttagtgtggttggcttagcatttgctgttattggtgataagttgttgcaa

SEQ ID NO: 15 - DNA, 290 bp, Tetranychus urticae
tcctcttcatcgtcaccgaaacagtcaatgttgggtgcaacccattgaggcatggctggctttcgccgtgaacttgaacttgtaactatggcaggtttacgtcgtgaactacccgttgaagggaaccctttggttgacccaaagccagtcagtgagggtctcaccgagacaccagctgaagcaatggttacacttatggttggtgctggtctagaggaaaggtcaagctcacctgagacgggtgatgaggcgtttgttgatattgcgttcaatgtgatggttgtggttgt

SEQ ID NO: 16 - DNA, 275 bp, Tetranychus urticae
tttacctttacggggaaccaatgatttgagaagattgatacaattctcatcggctgcccaggcgtgcatcttacggtacgagtcacgacctttttccgttaatttgtcccacggtttgggcttggagagaagctctgaaacggttccttgggatagtcctaaaacaaaacgggcaaacaaccgttgaccaatattgtgtattgataaaagttcacgaacacagcgagaaatgtttaaagtgttgagcgattcgttcataaatttgtccacatttt

SEQ ID NO: 17 - DNA, 172 bp, Tetranychus urticae
aatgttttctttgtgcaagtttcttatccaatccccagaagacttgaacaatacccagagtagctgaggttacgggatcggagaagtcaccaccgggtggtgatacagcacctacaatggttacacttccttctctttctggattacctaaacattttactcttccagctct

SEQ ID NO: 18 - DNA, 254 bp, Tetranychus urticae
aacataaccataaacatcaccaccggtaatgtgactaccaattctgacatattgagggaaaaagtcccatttgatgtctctgtttaaagagggtgtattgactccctttggaatgtaaatactttgggtaagatcatgaatatctttgagaggcctttgaataccatcaaaaatagatcccataatacctggtccgagctcaacagataatggtttaccggttcggagaactggatcaccaacagttacaccag

SEQ ID NO: 19 - DNA, 177 bp, Tetranychus urticae
gatgtttcttcataaacttgaatggttgctaaatctgtttcaagacgaataatttctcctactaattcctcatgaccaactctgaccaattcgtacatagcagcaccagccattttttcagcaataacgacacaacctgaaactccttggacatatccgtataatttttcattttcg

SEQ ID NO: 20 - DNA, 181 bp, Tetranychus urticae
cacaaacataacttggcctaaatctccagaaactttcaagtctcttacgtccataggatctccacgataagggacgataacttgagttcctcttctggttaaattgctgaccaaaactcggcctaatggacatcttgctccgaaaacagtaacaacattaccattaaacgatgatcttcct

SEQ ID NO: 21 - DNA, 359 bp, Tetranychus urticae
aacatctttagccatttgactggctgttgctggtgttccatcatcatattcaattaatccattatacaaagcttgggccatagcaattgcgttaccctgcatgtacaaattaaagtgttgattctctcctagctcaactcctgcaggtggatcagcataacctcgaagtaaactgaaaacataatcttcatcaccctctcttcctacagtaatattcgaaagatctggtggataggctcctccattagcgactcgagcagcttcttcattgggataaggacttggtatgtaatccgccaatttaccaggtctttgaaacattttaccattggcatcaggtccatctgtaatcgtggcct

SEQ ID NO: 22 - DNA, 247 bp, Tetranychus urticae
tgggctactgataccgagttcttccataactggtttcaattcctggagcaagtatgggtagatgtctttatcgtggttgtctgccttccatttaacagcttctaagtatcgtacacatagtgaatggtcattcactcgtcgacaggctctgaatgctgcttcaataatcctgggttcaggtataagatccatacccataagttcgtttaatgcttttcggatctcccatccatctatgtcaggccta

SEQ ID NO: 23 - DNA, 175 bp, Tetranychus urticae
attgtttttggttatggaggaatcgatattcacttgtgtcacgaggaacaatatcggtaagatcaagtccgtctttcacttgaggcacacctgacaggcaaatcagataaattacacagctaacaatcatcaccaaaaatttaaaaaatcttccatggaataaaggtaaataaat

SEQ ID NO: 24 - DNA, 470 bp, Tetranychus urticae
cctgatttttagtaagcccataaatccaaaaatttggacactagttagagctagaacaaatattaacaacgaggaggcccaacaattgaccaagagagcacaaaaggcaacaaaaatagctgcaaaagcgacgatcaaagatgcacctaatgaaagtctcaaacgaatatactgttcccaatatttgaatggaagtcctgttgggaagttcggcaggcctttttcctcataaactttacaaattgtcctaatttcttgaattgttgaggtaatttcctcggttgagttcatatcatgcaagtaaaaaggtatttcagcataagttaaaggctgagattttggtatttttaaatcatagtcagatgctacatgaatccattgacgaggttctggatgaaaatttgcttgggatgctgaataagcaatcgcatcattggtgacccaggcagtcaaataattataaaatgctt

SEQ ID NO: 25 - DNA, 186 bp, Tetranychus urticae
tgaaactaaattatgatggtgtcgcttctttgccggaggtgaacaaggaggcgagggagttgagattcgattctcatattcaattggaatgagttcgccgggtggaccaaagtaaaataagagtaaaggatataagataagaccattccaactggagatggcaaccgctccagaaaaaatgtaaaa

SEQ ID NO: 26 - DNA, 203 bp, Tetranychus urticae
tatcatcaggtaaatgtgaggtagtttgagttggaatttgatgtggcccagtattggtcacagttgttgtttccaccaccaactcaggctgaacaacaatttcgtgagatgatttgtaagaaggtggttcttctgaaatagttgacaaggatatctcggattgcacacgaggataaatacgtcgtgaatatgaaactaaatta

SEQ ID NO: 27 - DNA, 272 bp, Tetranychus urticae
ggtatagtaatctcgggtcctaataatttagaaccttcccagaaacaatccaagggtgttattattgcgcaaggtattaatttatccaaagcggcatctataagttgctcttcaaaggctggaatactcaaagtgtagcatatatcttttaaacgccatgtcgttgaaaacatatccacactgacttgagtaccaactttcaaaacttctaaatgtgaaagtaaggcacttggatggagaatgttttgattgtcattgtttggtgtttgaat

SEQ ID NO: 28 - DNA, 268 bp, Tetranychus urticae
cttccaacacttgacgagctttctcgatggtccaatcaatatgaagaacacgagcattatcctgccagtaagcaaacatatcttgttccgccattaattggatgatagattgaagagcatgggctttcacaatacgaccattcttgtttttcttaattcctccaataataagttgttccggccagtgcatccatttggtggcgaaaccataacatcctccggacagttcataactaacatccggctcacggtttgtatgtttatttgg

SEQ ID NO: 29 - DNA, 183 bp, Tetranychus urticae
gagtatatgttttatattccatcagtttttcggttcctctatggagaagtccaatatgtggatccaattttatgatttgttcaccatcaatttcacaaataagtcgaagtacaccgtgagcagcagggtgctgaggtccaaagttgaatctagcattttggatcactttttcatgaggctttt

SEQ ID NO: 30 - DNA, 229 bp, Tetranychus urticae
gttggtcactttgaaaatacgaagtaaacggacacaacgaagaaccgaaactcctaaaggttccatccattgattcttggttaatataatttccgaaatggatccaataacaacgaaacaatcaaaacgattaaaaagagatacaaaataaccttgcaatccaagactgtacatcttcagaaacatctcaagggtaaaaagtatcacaaaaaatatattagcattatct

SEQ ID NO: 31 - DNA, 192 bp, Tetranychus urticae
gaaaaaagcttcagcaaagtctctattggttgctgcatacagaaaactgtagacacatgaatgacacagtgctaaataccatgttgaattcagccactcttgagccactggtgaataaatattaacaatggaggcgattattaacggtgtccacatgagaaatgatgatatataaacacaaatattagattt

SEQ ID NO: 32 - DNA, 195 bp, Tetranychus urticae
ccaccaacaccaacggatcctccacttgagccaccttctcgcctctgtgaatacatattaaggcctggtattatatgaactcttaagcccattggcccgtaacttggcccacgacgaggcccacctcctttcctttgccatgttacatgagatttacagcaacagtaatagaaaagtttgaaaaaagcttcagca

SEQ ID NO: 33 - DNA, 232 bp, Tetranychus urticae
taaagatcaccggttgtcttgtaagctcgcccagattgaaatgatcgagcaaaaggtccactagtacatgaggaaccacccccaaacgagtgtccaccgccgaatccgctgctgtgtccaccgaaagagaacccacccgaatggtgtccaaaggaacttccaccgccttgacttccaccaccacttgaagtgattcgcgttgaaaaactcgacgagccaccaacaccaacgg

SEQ ID NO: 34 - DNA, 262 bp, Tetranychus urticae
cacctttaactcctactggaacatcaaactcaaaatcttggtaatcgtcatatggttgagtttttcttaaatcccatttaataccactacctcgaagcattaccccgctgaatccatgattaagagcatcttcagctgaaatcactccaatatcaacggttcgagctttaaaaatttcactgccagttagcaattcgtcaatttcatcaatacgattaggaaattgagttgcccattgataaatgtcatccattaaacctaa

SEQ ID NO: 35 - DNA, 193 bp, Tetranychus urticae
cccaacctttaacaaaagaaagcctaagaatacatagtctccgcaaatcatcaacacctattccagcgacagctgacaaactaattgcaggagcaataccaccacatgatgccggtccaggtatatgacctgcaacggcagcaacttgagctgcagcagcggcttgtgctgaagcagcttgttgttgcatttg

SEQ ID NO: 36 - DNA, 216 bp, Tetranychus urticae
taacctcttgatccagtaaagcttcaatattaagtggcttcacaacttcagcttgaaattggggatgacaataagcttctcggatcttatcaaagagtagatttttaacagttccctgagatttcggactgatcttctgaggcttgtaactgatgtttagcgtaggaacgtcaccttcaccactatcaggtgcataacgaccagctaataaacgga

SEQ ID NO: 37 - DNA, 220 bp, Tetranychus urticae
ttgccattcagcatatttgacaggatggttgataatatttgcagtgattctttgtctcaatgctctcaagtcgtcttcgatgtcataacaaaatggttccagtccaaagttggccttaaccttttcacctggagtttgaagcccaattgttggatacaaagggatatttggcaaatctgtaaaagcaataccaagattgtggccattcttggtgaagaaa

SEQ ID NO: 38 - DNA, 208 bp, Tetranychus urticae
atggtaaccaagtttcagctagaccacgaacaactccctttgtcattagagctccaccaagagctaaaccagaggccaaaccataagttgtagcccttctagcgtcagcagtgtctttacttcgtgatgtcgggtcttgtgttgcctttttccggacctcagtgaaatcaggaaccttgatgtctgtatgagctaacctgatttggct

SEQ ID NO: 39 - DNA, 369 bp, Tetranychus urticae
caacaaatccatattcagtcaagatacgtcttaaatctggatgagcatagaaaaatactccaaacatgtcatgtacttctcgttcataccagttggcaccctcgaagacctcacaacacgaatcaattggtgtcatttcatctgtgtaagtcttaaccctgactctcgagttgaaatttaacgataacagattgtaaatgatctcaaatctgtatgctcttgaaggtacatccataccaccaatatcagcaatacttgtgaattgacaattgtgatgtccttgtaagaaagcaagaaccggaacaactccttcaggagcaatcataatttccaattcatcgacatgagtaacttgaatcttctgaatga

SEQ ID NO: 40 - DNA, 193 bp, Tetranychus urticae
tatttttttgcctcgggctgaggttctgcgactacagttcctgctactaaaggaatctcttcagctggtattgctattgctggattggctgggaaagtttcccaaggagtttccaagtcaaatttacggaattcttgggctaattcaagtggttcttcgataatgctacccaattcatcatcataacgatact

SEQ ID NO: 41 - DNA, 211 bp, Tetranychus urticae
acctgagaatctttgagacccaattcttcggccaatttctttctgtccggctttgagatgtacttttgaacttgaaatctcttctctaatccttgtctttgtgcatctgaaaagacggctcgtctcatcatcccacggcgaggtttacctcgagaactggacatccaggctagaggatagcctagggtcaggttgttgtgatgaggatgat

SEQ ID NO: 42 - DNA, 174 bp, Tetranychus urticae
ttgttgtggttgtcgttataacctgaccctctgatgatgaggatgtatttttccttgagagcacgaccattccactgccaccgattcctcgagcaccacctgaaaacccattaccagagctgcccttggaagcaccgacagttgataccaggaaaagtgtggttaaatcgcgat

SEQ ID NO: 43 - DNA, 209 bp, Tetranychus urticae
ttcacttttaggttgctgtggatgaattgaagttccggatggattagaggttaatctacgcggattggaaccagaaccagatatggagaggttaccaggagatttggcaatggtggaaccactggaggttgaaccttgttcaaggctgtgttgatggttaaactgattggaatggcgtgttttaacattgtaatgatgtgattgaaatt

SEQ ID NO: 44 - DNA, 176 bp, Tetranychus urticae
ttgaacccaatccatctgaattgggtgttggaaggaaagctgaattttcctcttttgtaatgttcatgtaaaaacagatatcagatgctttcaagatgtaacttggaccaggatttaacatgatgggccaggttcccttcatatcagtttgaactccaattaaggccactccatat

SEQ ID NO: 45 - DNA, 245 bp, Tetranychus urticae
ctgtcgattgaaccctgcatccagtaaacaagaggaaaccatgagatggcatcaatgaaagcttgggttggttcggtttcaagtaggataatgattggttgcaaggatttaactgaatgaaagtgggctcgaagtggaactataaagttataaattgcatttgaggcaaaatcggcagccaagatgatacaacgagtaggccagttgtattcacgggcatgttgatgggaacaatgttcacaatc

SEQ ID NO: 46 - DNA, 335 bp, Tetranychus urticae
ataagcaggaggaggttgattgggttcacctggtgctgagttcagttgaagtgaaggtacaatggtaatggttggaggcaagtgaagttgaaagtctccgagttcctcagctgttcgctcttcggaaatactttgatgtctacttgagccactgccaagtgatcttaaatcatcacaatttaatcttgaatctttcacttgatcagtatcagtcattgtcgaggttttaggttgcaactgttgcaccggctgtgcctgtcctggtgaaccagaagctggacttgcattggcattagaggttatcgaaccagttcgacttctttttcgtttaagtg

SEQ ID NO: 47 - DNA, 263 bp, Tetranychus urticae
taatgggcaaaggaatgggcgagggttcacgagagatttcctttgatcgactgtaccttcgacctcgaccaccgtgttttttacaataggaagcaattggtgcgggcaggttaatggtgttgcttgggcttaaagaggtgttcattgaggaggaggacaaaggtccaggggcttgtaaaaggtcaaccaaggcttcttgttgactgaactctttttcccagtccagttggtggagtcgtatatacaaaaagattgaaaaggtt

SEQ ID NO: 48 - DNA, 283 bp, Tetranychus urticae
taaactggagtggttcgccgtaaattgtccaaaggaaattccttggtcggtgaatagtctcccatgtattcactgttgctgctaaaatcttgttccagtattggtgtgggataagaggctcggcagattgagtcatgaggcggtgaataatcttcacgggaagatgattctctgtccatatcattggaaccagaaactgttgtgtgatgatgtgaggaagattgtgtttgactgagattggattcacgatgacagttgtaatcatgttgctgctgttgagatg

SEQ ID NO: 49 - DNA, 470 bp, Tetranychus urticae
gagcatcggaggtgtcaaaaaaaataaacccaagagaggcaacacgatagttaatggagacaaaaatgattgactcttcaccagccaaaacggaaccatcgtaaaaatcaagtgacgagcttccagaccaaaagcttcctccatagatccataataaaacggctgctgatttgggacgaggtcgaggtacccaaatgtttactgataggcaatcctcgtcaaggggaacatttggattccattcggtggcacccataaagttaccaaaaaaggtatcattgacttggtaacatgcacctgaaaatgaggttgcattgaatattccttgccatgaatcgatgggcttaggatggcgaaagcgaaactttccagtcggtggttttgcataacggatacccaaaaaagcatcaactggtttaccggtgggtgaaacaactgatcgaccacgaacataacctttttttgtcaac

SEQ ID NO: 50 - DNA, 295 bp, Tetranychus urticae
tcataatcatcctcttcgccatagagcaaatcgatcagaactttatctgcatttatggctatcttgacatcctgtgcattaataaaactatgacgtatatttttaacataattgcctgctaatttgagaacatcagcagcaatgtattcgaccagactgcaaaggtaaatggtcagtggcatctcaatgcgataagggaagacttcctttgaaaggagatcatgcatcttctcaatgctgtgttttgtcttcaaaggacaattacctaaaggatcacgattattatttatgctat

SEQ ID NO: 51 - DNA, 185 bp, Tetranychus urticae
caaattcaacattttcggccatctcttcaaaacacattccgattccaatggtttcagagttatctccaccgttcatttccatagcatcttcaacaagtccgagaaaattgacactgaactcgtagatgtcgatgatattagaaaaaatatcatcaatgtctttctcactaattaatggcttatcc

SEQ ID NO: 52 - DNA, 165 bp, Tetranychus urticae
attttggtaaaatatacttggcagaaagaataaacttatgcccagcagtatccagaacattaattattggttgttgttttagcaaggtaacaagtctagcttttggtacagtttcaggtgcgtcttgtcgaagcatatcaagggtatatttttcaaatacttcaa

SEQ ID NO: 53 - DNA, 211 bp, Tetranychus urticae
ttttgttcaacatatttcttttgtttttactccgctgattacttcgattgaggcgtaagtaaccttcgtaaatcttcaagttacgattacgagttgcttcgttgatttgtttctgcattggagttagacatccctcagtttgtttgaagctctctttgtctgcttctgtcgttgcaaggctcatcagtaacttcatgtaatcaaaatagtg

SEQ ID NO: 54 - DNA, 216 bp, Tetranychus urticae
cagttcaaagtcacaattctctttaccattgggtggcaaatattcgataggcggaggtacttcagataagattttgttttcggaatttttgttggattgcttttgctgttttcttgagatcatattggacaatgtttggacccatttgaccacattacttttttccttggacactttttctttgaggaaaattcttaacgattcaagtagttgatc

SEQ ID NO: 55 - DNA, 173 bp, Tetranychus urticae
caacgatttctctctccaaccaataagtgaaattgttggaatgtctgctaattttaagcagattagcactattaatttctttaccctcttttatccatttcaggcctatcaactcggacggttggacagcacaataaagatcaaactcgtacaaagtcaattgcctggcaaat

SEQ ID NO: 56 - DNA, 197 bp, Tetranychus urticae
aatcaattttttatgcttaggatggagagcattcaaggtatgttccaatcgatgaacatgagcagattccaaagctgacattatttcgatgattccactaaaattgttcagatctgaaagtacttgcataatatcaataattcggctcattagggcaactctttcttcaaagttgaccgtttcaacgatttctctct

SEQ ID NO: 57 - DNA, 294 bp, Tetranychus urticae
ttgttgttgttggtgaaacagttgcacttgtaaccgatgactcagagttagtcctttggtggaaatttcgagttctcggtggtattggcggtggtgttattgttgatgcagaaatgattggcggaggtgttggtgcaggagttggcaaattgccgatgatcacattggcgaaaacagaggtatcagaagttgttgatggcggagttttgggtgaaattagagttggtgaagttggattagaagatgatgtactctgtggatgattggccataaatgttgatgtgggtaatgatg

SEQ ID NO: 58 - DNA, 172 bp, Tetranychus urticae
ctgaagagtgaaatgctgatgatcgggtcgaagaaaagttggttatcgaagcatacgagtttcgaggttcaccagcatctacacgttgacgagctcgtttcactttacctgttgaatttgtgtttgaggttcgattggaaggtgactctgacttgtggtgatgatgatggga

SEQ ID NO: 59 - DNA, 174 bp, Tetranychus urticae
ttacagctaatattgttctctttgtattgtgttaacattttatcgttaaattgaacaccgataagaccagcagaaccgcccgaaccaggtaatgatgatagattattgggttgattattggggcaaattgaagacgatgaacgtggtagggcgtaactagatgatggtgacttt

SEQ ID NO: 60 - DNA, 175 bp, Tetranychus urticae
tgtttgactgccgatgagactcttgacttttggacattttctcttgatagacgagtttacgagcttcattcaacaactgacctggaccttggatcccgaaacgatacgaaccgccaaactgtgagcttggtgttgctcgaggctcggaaaggtcgtcttcttcattcaacaaatc

SEQ ID NO: 61 - DNA, 235 bp, Tetranychus urticae
cagagtcaattggtgaaccttcccttgcactacaattttgattatcacgtcctcctaaaaagctaagagatcctgttggagttgagacgggcgttgattgatcccgaccagggttgttgcttgaaccagcagccaatgcattatcatttggtccaacaatatcagatcctgatgaccgttgtaatgattggagttgctgttgtgccatttgtgcttgatgttgctgtgcctgttg

SEQ ID NO: 62 - DNA, 407 bp, Tetranychus urticae
ctgccgctgctgcagccgcagcttggcataaaagagactcacgaaggaaccttgcgtgatcagagaaaggaaaagccgggccaccgaatagttcggctgaatgaagacccggggttgggattgccactgaagttggaagtgaaactggtaccaaagggggaggaggggcagttggaggcccaatgggaggaccagtgtgaggagataaaccaaaattaagagcaggaggaggagaggcaaggtcacctcgggctatcaaatttgctaaaagaggttcttgattaagaatacgactaactgttcttggtgttatccttgaatcggtaaccttatttcgtttccttttcggtgttaccacaagggacattgcttcggtttgttcacttgctaaatcactcaagtcacca

SEQ ID NO: 63 - DNA, 248 bp, Tetranychus urticae
ttgaagaggaattgaattgccgcaaaaggaaagtaaaggtgttgatgtgtcataaccagttgaatcattgagagattctgatccatcagcgccattctcagatcttgtaccgccaaattgactaagagactcgggtgatgctgtgtcaccaccgccagcggcacgattggcttgggctatcattgcaaggtgatttgctgccagagcattttgaatggctgctgctgtggctgcttgatgatgatgat

SEQ ID NO: 64 - DNA, 176 bp, Tetranychus urticae
gcaccaacactgaaataaccccaaacatttttgttgctcctaaaattgagctcttgaaccgttccacttgatggcttgaaaccttcatcaggattttctgatgtgattcgggctgcaataacatggccccagggttgagcacgtcctaatttgaagtcaattggattatcattgag

SEQ ID NO: 65 - DNA, 251 bp, Tetranychus urticae
tacattttcgtagattagttcaacattaacggaatttgttaaagtcgtagcatgcggaatttggcctttctgtaaagaactttcaaaattttgccaattaccagaaaattgagcatcagcaacatgaatagaaccacaaataagggcaagacttgtcttaggcttgtcactttgaacacgttcagagattaatttatctaaccagccagtgtcaatttcatttttctggaaagcttccgtttctaataaag

SEQ ID NO: 66 - DNA, 179 bp, Tetranychus urticae
tgattggcaacaattactcgatattggtcaatttcttctttcatgtaagtaacaattgaagatccatcaacagataagagtaaacccgaatctgacatttggtgaacttcaacctctttgtaggaatcattgagaactacaaaataactgttgggcccacttttagcaacattaaacaa

SEQ ID NO: 67 - DNA, 213 bp, Tetranychus urticae
gcagaatcagtttcactaggattttttcccacaatatcgcgagccatcaattcttgccaaccctctgagtataattcagctcttcttactttggttggatcatcaagatcaaggcgagccaagatacaacccgcttccagaactgaaccaaatcgtttagcatgtcgaattattccactttctgaagttgttaatgtcattatcattttcatg

SEQ ID NO: 68 - DNA, 205 bp, Tetranychus urticae
tttaagtaaatcttgaacacaacttcttaaacgaccacgaataccattacgataacgttgtactaattgaacaattccttgagtattgaggaaaaaaacatcacgatcacttcgtttctccaatgttgccgcataactgtcaatgacagaggcaatttcttgggacgggaatatttgtaacacagcggttatattcttggcatat

SEQ ID NO: 69 - DNA, 206 bp, Tetranychus urticae
caagagaagcttttctaacaactatattggaatggtagaagaattgatggagaacatcaaaaatagatgtctcactgataattaacttattcatattgtttgcatcaaattcatgtgagtacatatcaatgaccgagagaaacattgcttccatttgactaaatcgtagagcgtatggtggttggtgagcagagatgagtacctgt

SEQ ID NO: 70 - DNA, 188 bp, Tetranychus urticae
gttctaaatttttgaaggcagctattacacctattctgtaattggaatgtaaatgagcacttgattcgccagaaaaattatttgataagccctcggtattttgtgattgaggttgtgaggcaattgatggatgtgacgaaggaagtataaagtggaaaactgctgttggtataacagaatcattttgt

SEQ ID NO: 71 - DNA, 307 bp, Tetranychus urticae
aaagtcaacaacaagttctacataagataaaactgttttgggcatttcaagttcatagtcttctttagattttaaatattccctccatatttgtgtaagtgcggtttgaaacatgacaggaaaatcgtaaatataagtagttccgttactttgagcaagaaatcttttagattgtaaataatctttagttcggtatggagaggaaattggctggtcatttaagggtcctggttcataataaggtgcaccccatgcttcaaatttagttaatcctgaagcaggatcacgagtttccttgtaaagatga

SEQ ID NO: 72 - DNA, 451 bp, Tetranychus urticae
aaatcattaatggtaagccttcacgattgaaatcattgatagcttgagcagttttgaacgcactgtcagggaaccatacttgaccagcttgagattgaacttttgtttcggaagttggattagcaggatcagcaggaattataacttcagttttacgaacttctacagatataacacctacaggaataccaccaagacgagctcgcccaacaacaaccgttttagcccaacctgccataatttcatggaatgaacctttgtcaaagaaaccttcttcccaaactccttgcggtgattcgcgtccattcaaaatccatcgaggatcataaggcgaacttgtgggacaatagacaacatcacgatcaattatatcagtcggttccattatcggtaaaggcgctggctttattttagttaaattgaccaatttagttgccttttctcgtttttt

SEQ ID NO: 73 - DNA, 268 bp, Tetranychus urticae
caataatctttttacagataacgtcatttctgtgcatagcttttatttgatctttaattctaaatctgatttcgactgaaccttctggctcgataacaccaccacgactatcaggatcagcatacatctccatgtgtcgagggttaattgtggtatcaacaacagcccaagctccaccacgtaattcagcataaggaggaatataaacaataattggttgattgtaggctacaagagcatctacgatttgagcaccaaatttcagaat

SEQ ID NO: 74 - DNA, 227 bp, Tetranychus urticae
tattaacaatattattaacattggtaggattattggttggcattggcacattggccatgttggttgatgcaagttgctgatgagctgtgcagcacaagcaaagtgaggtacgattgcctatcaagttagtcaagtgatcgtcaagacctgttgagagaccttcatgacaacccaagtgaagggccccttcgcttgccgctgactgaatggtattccaatgttgctta

SEQ ID NO: 75 - DNA, 257 bp, Tetranychus urticae
tcattcgattggtaatgaatcgtatctctctgagaatggcattgatctcgcgacccttgcaaatgaatgaacaacaaggttgactttgtgaatagtcaacttgatgatggccgggggactcattgagcaacgatgtggtcccttcagtatcaccaagacccaaacctaggctagtcccacctctggctccatcattcaagtcctcggcttcaagtgaacctggctctagattaagttgatcacaaggtaaaccatta

SEQ ID NO: 76 - DNA, 181 bp, Tetranychus urticae
aattattgttgtcactaatttcgtgtaccgaatctaactcttcagctccaattggacgtccataaggactgtctgttttactgatatcttgaatggattcactttgccctggttcctttgattcatcaataacataaacttttacgttaggggaatcatttactttttgatgatggtgttg

SEQ ID NO: 77 - DNA, 233 bp, Tetranychus urticae
gataaatttgtcccaataacattcgtaaaatttattacagtctcctggatgttggaagagaccaggtcgagtacaagcagcttctggaactgaaccatcagtgaagtttcttaaaccaggataaaggtttgagttgtataagctttgtaaagagttaactttagctgcaggaagatgtccattatccgtattgtaagaatcgccagagtatgatgaatcatcttcatattcaa

SEQ ID NO: 78 - DNA, 229 bp, Tetranychus urticae
aaggtaagagttgaaaacaaatccaagtgcattaagaagggaggcaattgtatctgtacaggcaaggttaagcgagagtattagaacgggcgacttcttatgtgttcccatacgtcccacaatgatgaggccaacattgagagtgacggaaacgagacaggcaaatatcaatggaggacaaagcaatttgtaaagtgtaatctgagtttgtctttctgcatcatcttca

SEQ ID NO: 79 - DNA, 249 bp, Tetranychus urticae
atttttgttttcaaagatatcgtggatacaggttgccagtttacggctaacactctcaacataagcatcgagtttagcaagatcatctgagagtccaacaagagtgtctaatgtaccaactttgaggtcaggaatgggaaatttataattcttggctaaatcctgcttagaagtgacattattcaagctttcccaagtttgatggattgttttctctccaggagctgagatgagccaaaattcactcat

SEQ ID NO: 80 - DNA, 191 bp, Tetranychus urticae
caaatttacactctcgagcgcgagttttaaactcatcaatacatttggagaaaagggttactgtgaatagaccatgctcattgtcctcaaagattaattccgatgacctgggagcaacgtaagcagctagtttttcgtacttcgcattccattctttgaagtatgctttaggaacaacaaccagcaaagtg

SEQ ID NO: 81 - DNA, 240 bp, Tetranychus urticae
tcttctttgttgtttcttattgggatgtataagtacagcaagaaaattaactggtaatccatatcgtaaaacagattcaacgaaaactctcaaagctttcacatggacccaagcaataaaggcttcagaaaaattgatcttcagccatctcagtaaattttggaatttcttctctttatcttgttctaatttaaccagttgatttttactatctgcaatttcttgttcattgtattggaa

SEQ ID NO: 82 - DNA, 329 bp, Tetranychus urticae
caaggaggtttcatcaggatacgtgtaagaacagctaacctttcctctaaagctttcttttgttgtaaagcatcttgaaggttgtttaataattctctttctgagcttcgccagagagaaataatatcttccttgctcatggattgaattttaacagtgtcaaccatactggtgtgacgtggacttggtataatcatggaggctcgaggagattgggaataaagaagggtcattttatcttgttcacttggtgaatttgctggtatactaagttgctgtttaagaatagagagatcatcgaaagaccaggttttaattatgttcatctc

SEQ ID NO: 83 - DNA, 226 bp, Tetranychus urticae
tgtttcaatgttgattccaatgcactaatcacagcttcaccccaatcagatcgagatgtaccaaactccattgtcattgggttaacaatgtcaacccttaaaccaaccgaggcagctgctttcaacaaatttacacccaattcaggagttaaaccataatcatcgccttgaaccattaaagttaaccgaacagtcgtttcaggtctagcattttgtacatttttca

SEQ ID NO: 84 - DNA, 180 bp, Tetranychus urticae
aaattttggtgaattcatattcagactgggcaattgaactgcacgatggtgaaatacctccacctggacatttaccattatcacgtccaacagaccaaaagccgagatatccaagtccagtttttttagcatagtcgattaatgatcgagcatgtttaacctcaaagatttttccattaa

SEQ ID NO: 85 - DNA, 218 bp, Tetranychus urticae
ataaaatctccaagcatagtacgagttccttcggatctgaatttatcctcccaaaatggttgatcgacatcatgaacgtacttataattcatcaaggtattagttgtagtgagcttacgagaggaaacatccgacataggatttaccattccaactgaagacatcagcccattacggaaaggtaacaaaattgaaggtcgagtgactgtggttaacat

SEQ ID NO: 86 - DNA, 556 bp, Tetranychus urticae
atcagcagcagcaacagcagcagcaacaacaccaacaacaacaacaccaacagcaccaacaacaacagcaacaagctcaagtccaacaaactctttcaaatcaacagcaatcacttgatactcaaccaggagttggtctatcctcgggttcacctttcatcaaaggtgaaggttacattcctcaacattcccctgaggtaccaagtgaaagccatacaccattacattcatcacttggtccaaatccgggttccaatggtgtcaactcaaatggccctggtgcacctgttaatagtatcggtaatgcaaacctaggaaactcccttgtatcctcgatagtctcctcttcatcttcttcttcctcttcctcaacagcctctggtcctgttcttactcgttccccggttcttccggtttactctgaatcaccacttaactcatcacaaacgattgctcgaagttcacctggaaccattcattcaccttcccacactcccaaccaatggtcctcaatgggtcagataacaccaaaacaagagatcggtcttggcctggg

SEQ ID NO: 87 - DNA, 470 bp, Tetranychus urticae; identical to SEQ ID NO: 3

SEQ ID NO: 88 - DNA, 27 bp, Artificial sequence
  Primer
ataaaatctccaagcatagtacgagtt

SEQ ID NO: 89 - DNA, 24 bp, Artificial sequence
  Primer
ttaaccacagtcactcgaccttca

SEQ ID NO: 90 - DNA, 25 bp, Artificial sequence
  Primer
tgattgaattcactttttcgcacat

SEQ ID NO: 91 - DNA, 27 bp, Artificial sequence
  Primer
aaataactgaatctggccaagttatta

SEQ ID NO: 92 - DNA, 24 bp, Artificial sequence
  Primer
ctaaaaatctaattgcagtggtag

SEQ ID NO: 93 - DNA, 19 bp, Artificial sequence
  Primer
cgtttatctggcaatggag

SEQ ID NO: 94 - DNA, 28 bp, Artificial sequence
  Primer
aatgttttctttgtgcaagtttcttatc

SEQ ID NO: 95 - DNA, 24 bp, Artificial sequence
  Primer
gctggaagagtaaaatgtttaggt

SEQ ID NO: 96 - DNA, 20 bp, Artificial sequence
  Primer
acctgagaatctttgagacc

SEQ ID NO: 97 - DNA, 22 bp, Artificial sequence
  Primer
atcctcatcacaacaacctgac

SEQ ID NO: 98 - DNA, 28 bp, Artificial sequence
  Primer
taacctcttgatccagtaaagcttcaat

SEQ ID NO: 99 - DNA, 24 bp, Artificial sequence
  Primer
gtttattagctggtcgttatgcac

SEQ ID NO: 100 - DNA, 21 bp, Artificial sequence
  Primer
caaggaggtttcatcaggata

SEQ ID NO: 101 - DNA, 28 bp, Artificial sequence
  Primer
atgaacataattaaaacctggtctttcg

SEQ ID NO: 102 - DNA, 20 bp, Artificial sequence
  Primer
ctgtcgattgaaccctgcat

SEQ ID NO: 103 - DNA, 24 bp, Artificial sequence
  Primer
tgtgaacattgttcccatcaacat

SEQ ID NO: 104 - DNA, 27 bp, Artificial sequence
  Primer
taagcataataagttctgataacatcc

SEQ ID NO: 105 - DNA, 23 bp, Artificial sequence
  Primer
tctttgaatgttgagtcggaatg

SEQ ID NO: 106 - DNA, 25 bp, Artificial sequence
  Primer
cacaaacataacttggcctaaatct

SEQ ID NO: 107 - DNA, 27 bp, Artificial sequence
  Primer
aagatcatcgtttaatggtaatgttgt

SEQ ID NO: 108 - DNA, 24 bp, Artificial sequence
  Primer
ccactgttggtgtaagttgtgaat

SEQ ID NO: 109 - DNA, 23 bp, Artificial sequence
  Primer
ttcaatcacttgtcgatatgagc

SEQ ID NO: 110 - DNA, 22 bp, Artificial sequence
  Primer
tggattgttgatggttagactc

SEQ ID NO: 111 - DNA, 18 bp, Artificial sequence
  Primer
gctgctgcggctgcaact

SEQ ID NO: 112 - DNA, 24 bp, Artificial sequence
  Primer
tgattggcaacaattactcgatat

SEQ ID NO: 113 - DNA, 27 bp, Artificial sequence
  Primer
tttaatgttgctaaaagtgggcccaac

SEQ ID NO: 114 - DNA, 20 bp, Artificial sequence
  Primer
tgggctactgataccgagtt

SEQ ID NO: 115 - DNA, 21 bp, Artificial sequence
  Primer
gcctgacatagatggatggga

SEQ ID NO: 116 - DNA, 21 bp, Artificial sequence
  Primer
tgagatgagtatttacagggg

SEQ ID NO: 117 - DNA, 24 bp, Artificial sequence
  Primer
ttacgttcttcctcctattcttca

SEQ ID NO: 118 - DNA, 28 bp, Artificial sequence
  Primer
aattattgttgtcactaatttcgtgtac

SEQ ID NO: 119 - DNA, 27 bp, Artificial sequence
  Primer
caccatcatcaaaaagtaaatgattcc

SEQ ID NO: 120 - DNA, 23 bp, Artificial sequence
  Primer
atggtaaccaagtttcagctaga

SEQ ID NO: 121 - DNA, 25 bp, Artificial sequence
  Primer
caaatcaggttagctcatacagaca

SEQ ID NO: 122 - DNA, 24 bp, Artificial sequence
  Primer
aacataaccataaacatcaccacc

SEQ ID NO: 123 - DNA, 24 bp, Artificial sequence
  Primer
gtgtaactgttggtgatccagttc

SEQ ID NO: 124 - DNA, 24 bp, Artificial sequence
  Primer
caacaaatccatattcagtcaaga

SEQ ID NO: 125 - DNA, 26 bp, Artificial sequence
  Primer
ttcagaagattcaagttactcatgtc

SEQ ID NO: 126 - DNA, 27 bp, Artificial sequence
  Primer
cctgatttttagtaagcccataaatcc

SEQ ID NO: 127 - DNA, 28 bp, Artificial sequence
  Primer
cattttataattatttgactgcctgggt

SEQ ID NO: 128 - DNA, 28 bp, Artificial sequence
  Primer
gataaatttgtcccaataacattcgtaa

SEQ ID NO: 129 - DNA, 27 bp, Artificial sequence
  Primer
aatatgaagatgattcatcatactctg

SEQ ID NO: 130 - DNA, 19 bp, Artificial sequence
  Primer
ataagcaggaggaggttga

SEQ ID NO: 131 - DNA, 25 bp, Artificial sequence
  Primer
ttaaacgaaaaagaagtcgaactgg

SEQ ID NO: 132 - DNA, 27 bp, Artificial sequence
  Primer
cagttcaaagtcacaattctctttacc

SEQ ID NO: 133 - DNA, 28 bp, Artificial sequence
  Primer
caactacttgaatcgttaagaattttcc

SEQ ID NO: 134 - DNA, 22 bp, Artificial sequence
  Primer
caacgatttctctctccaacca

SEQ ID NO: 135 - DNA, 24 bp, Artificial sequence
  Primer
tgccaggcaattgactttgtacga

SEQ ID NO: 136 - DNA, 19 bp, Artificial sequence
  Primer
tgtttgactgccgatgaga

SEQ ID NO: 137 - DNA, 25 bp, Artificial sequence
  Primer
ttgttgaatgaagaagacgaccttt

SEQ ID NO: 138 - DNA, 20 bp, Artificial sequence
  Primer
atgaatgcttttgccaacgg

SEQ ID NO: 139 - DNA, 27 bp, Artificial sequence
  Primer
gttaatatttgttctagctctaactag

SEQ ID NO: 140 - DNA, 27 bp, Artificial sequence
  Primer
aatcaattttttatgcttaggatggag

SEQ ID NO: 141 - DNA, 25 bp, Artificial sequence
  Primer
gagaaatcgttgaaacggtcaactt

SEQ ID NO: 142 - DNA, 22 bp, Artificial sequence
  Primer
taatgggcaaaggaatgggcga

SEQ ID NO: 143 - DNA, 28 bp, Artificial sequence
  Primer
cttttcaatctttttgtatatacgactc

SEQ ID NO: 144 - DNA, 27 bp, Artificial sequence
  Primer
tgaaactaaattatgatggtgtcgctt

SEQ ID NO: 145 - DNA, 22 bp, Artificial sequence
  Primer
tacattttttctggagcggttg

SEQ ID NO: 146 - DNA, 24 bp, Artificial sequence
  Primer
caagagaagcttttctaacaacta

SEQ ID NO: 147 - DNA, 22 bp, Artificial sequence
  Primer
ggtactcatctctgctcaccaa

SEQ ID NO: 148 - DNA, 23 bp, Artificial sequence
  Primer
ttgaacccaatccatctgaattg

SEQ ID NO: 149 - DNA, 21 bp, Artificial sequence
  Primer
tggagtggccttaattggagt

SEQ ID NO: 150 - DNA, 25 bp, Artificial sequence
  Primer
aatttgtccacattttgtcgtaaag

SEQ ID NO: 151 - DNA, 25 bp, Artificial sequence
  Primer
caacaacttatcaccaataacagca

SEQ ID NO: 152 - DNA, 24 bp, Artificial sequence
  Primer
gttctaaatttttgaaggcagcta

SEQ ID NO: 153 - DNA, 27 bp, Artificial sequence
  Primer
aaatgattctgttataccaacagcagt

SEQ ID NO: 154 - DNA, 23 bp, Artificial sequence
  Primer
ggtatagtaatctcgggtcctaa

SEQ ID NO: 155 - DNA, 24 bp, Artificial sequence
  Primer
caaacaccaaacaatgacaatcaa

SEQ ID NO: 156 - DNA, 25 bp, Artificial sequence
  Primer
ttgttgttgttggtgaaacagttgc

SEQ ID NO: 157 - DNA, 24 bp, Artificial sequence
  Primer
cattacccacatcaacatttatgg

SEQ ID NO: 158 - DNA, 18 bp, Artificial sequence
  Primer
gagcatcggaggtgtcaa

SEQ ID NO: 159 - DNA, 24 bp, Artificial sequence
  Primer
gacaaaaaaaggttatgttcgtgg

SEQ ID NO: 160 - DNA, 26 bp, Artificial sequence
  Primer
ctgaagagtgaaatgctgatgatcgg

SEQ ID NO: 161 - DNA, 21 bp, Artificial sequence
  Primer
catcatcatcaccacaagtca

SEQ ID NO: 162 - DNA, 21 bp, Artificial sequence
  Primer
cagagtcaattggtgaacctt

SEQ ID NO: 163 - DNA, 18 bp, Artificial sequence
  Primer
caggcacagcaacatcaa

SEQ ID NO: 164 - DNA, 26 bp, Artificial sequence
  Primer
cccaacctttaacaaaagaaagccta

SEQ ID NO: 165 - DNA, 21 bp, Artificial sequence
  Primer
atgcaacaacaagctgcttca

SEQ ID NO: 166 - DNA, 21 bp, Artificial sequence
  Primer
tcataatcatcctcttcgcca

SEQ ID NO: 167 - DNA, 27 bp, Artificial sequence
  Primer
gcataaataataatcgtgatcctttag

SEQ ID NO: 168 - DNA, 26 bp, Artificial sequence
  Primer
tgtttcaatgttgattccaatgcact

SEQ ID NO: 169 - DNA, 25 bp, Artificial sequence
  Primer
aaaatgtacaaaatgctagacctga

SEQ ID NO: 170 - DNA, 28 bp, Artificial sequence
  Primer
aaagtcaacaacaagttctacataagat

SEQ ID NO: 171 - DNA, 26 bp, Artificial sequence
  Primer
tctttacaaggaaactcgtgatcctg

SEQ ID NO: 172 - DNA, 26 bp, Artificial sequence
  Primer
aacatctttagccatttgactggctg

SEQ ID NO: 173 - DNA, 22 bp, Artificial sequence
  Primer
ccacgattacagatggacctga

SEQ ID NO: 174 - DNA, 26 bp, Artificial sequence
  Primer
ttgaagaggaattgaattgccgcaaa

SEQ ID NO: 175 - DNA, 19 bp, Artificial sequence
  Primer
atcatcatcaagcagccac

SEQ ID NO: 176 - DNA, 26 bp, Artificial sequence
  Primer
ttgccattcagcatatttgacaggat

SEQ ID NO: 177 - DNA, 19 bp, Artificial sequence
  Primer
cttcaccaagaatggccac

SEQ ID NO: 178 - DNA, 24 bp, Artificial sequence
  Primer
ttgttgtggttgtcgttataacct

SEQ ID NO: 179 - DNA, 22 bp, Artificial sequence
  Primer
gcgatttaaccacacttttcct

SEQ ID NO: 180 - DNA, 23 bp, Artificial sequence
  Primer
tcctcttcatcgtcaccgaaaca

SEQ ID NO: 181 - DNA, 21 bp, Artificial sequence
  Primer
accacaaccatcacattgaac

SEQ ID NO: 182 - DNA, 27 bp, Artificial sequence
  Primer
aaggtaagagttgaaaacaaatccaag

SEQ ID NO: 183 - DNA, 25 bp, Artificial sequence
  Primer
agatgatgcagaaagacaaactcag

SEQ ID NO: 184 - DNA, 28 bp, Artificial sequence
  Primer
tactccactagagttatatcatgagtct

SEQ ID NO: 185 - DNA, 24 bp, Artificial sequence
  Primer
aatggacgatgaactggttaaatt

SEQ ID NO: 186 - DNA, 24 bp, Artificial sequence
  Primer
accaataaacatttccttgtggtg

SEQ ID NO: 187 - DNA, 22 bp, Artificial sequence
  Primer
cgagaaatttttggctcgtgat

SEQ ID NO: 188 - DNA, 26 bp, Artificial sequence
  Primer
caaatttacactctcgagcgcgagtt

SEQ ID NO: 189 - DNA, 26 bp, Artificial sequence
  Primer
tttgctggttgttgttcctaaagcat

SEQ ID NO: 190 - DNA, 24 bp, Artificial sequence
  Primer
aaatcattaatggtaagccttcac

SEQ ID NO: 191 - DNA, 25 bp, Artificial sequence
  Primer
aaacgagaaaaggcaactaaattgg

SEQ ID NO: 192 - DNA, 26 bp, Artificial sequence
  Primer
acaagtgattgaattgaatcgacaaa

SEQ ID NO: 193 - DNA, 22 bp, Artificial sequence
  Primer
caatgtgaaccaaaacacctct

SEQ ID NO: 194 - DNA, 24 bp, Artificial sequence
  Primer
tatttttttgcctcgggctgaggt

SEQ ID NO: 195 - DNA, 24 bp, Artificial sequence
  Primer
atcgttatgatgatgaattgggta

SEQ ID NO: 196 - DNA, 21 bp, Artificial sequence
  Primer
tttacctttacggggaaccaa

SEQ ID NO: 197 - DNA, 28 bp, Artificial sequence
  Primer
atgtggacaaatttatgaacgaatcgct

SEQ ID NO: 198 - DNA, 27 bp, Artificial sequence
  Primer
tcattcgattggtaatgaatcgtatct

SEQ ID NO: 199 - DNA, 27 bp, Artificial sequence
  Primer
tggtttaccttgtgatcaacttaatct

SEQ ID NO: 200 - DNA, 21 bp, Artificial sequence
  Primer
cgagtcgaggttgacccacag

SEQ ID NO: 201 - DNA, 28 bp, Artificial sequence
  Primer
atttttgtctccattaactatcgtgttg

SEQ ID NO: 202 - DNA, 24 bp, Artificial sequence
  Primer
tcttctttgttgtttcttattggg

SEQ ID NO: 203 - DNA, 27 bp, Artificial sequence
  Primer
caatacaatgaacaagaaattgcagat

SEQ ID NO: 204 - DNA, 22 bp, Artificial sequence
  Primer
taaactggagtggttcgccgta

SEQ ID NO: 205 - DNA, 20 bp, Artificial sequence
  Primer
ctcaacagcagcaacatgat

SEQ ID NO: 206 - DNA, 28 bp, Artificial sequence
  Primer
aaattttggtgaattcatattcagactg

SEQ ID NO: 207 - DNA, 28 bp, Artificial sequence
  Primer
atggaaaaatctttgaggttaaacatgc

SEQ ID NO: 208 - DNA, 21 bp, Artificial sequence
  Primer
cacctttaactcctactggaa

SEQ ID NO: 209 - DNA, 27 bp, Artificial sequence
  Primer
ggtttaatggatgacatttatcaatgg

SEQ ID NO: 210 - DNA, 18 bp, Artificial sequence
  Primer
cttccaacacttgacgag

SEQ ID NO: 211 - DNA, 23 bp, Artificial sequence
  Primer
aataaacatacaaaccgtgagcc

SEQ ID NO: 212 - DNA, 23 bp, Artificial sequence
  Primer
taaagatcaccggttgtcttgta

SEQ ID NO: 213 - DNA, 18 bp, Artificial sequence
  Primer
ttggtgttggtggctcgt

SEQ ID NO: 214 - DNA, 23 bp, Artificial sequence
  Primer
caaattcaacattttcggccatc

SEQ ID NO: 215 - DNA, 27 bp, Artificial sequence
  Primer
taagccattaattagtgagaaagacat

SEQ ID NO: 216 - DNA, 26 bp, Artificial sequence
  Primer
tacttggtgcacttgtaacaatacgg

SEQ ID NO: 217 - DNA, 19 bp, Artificial sequence
  Primer
taaccacaggcgatatgag

SEQ ID NO: 218 - DNA, 32 bp, Artificial sequence
  Primer
atttttgttttcaaagatatcgtggatacagg

SEQ ID NO: 219 - DNA, 22 bp, Artificial sequence
  Primer
agtgaattttggctcatctcag

SEQ ID NO: 220 - DNA, 29 bp, Artificial sequence
  Primer
attttggtaaaatatacttggcagaaaga

SEQ ID NO: 221 - DNA, 29 bp, Artificial sequence
  Primer
aagtatttgaaaaatatacccttgatatg

SEQ ID NO: 222 - DNA, 25 bp, Artificial sequence
  Primer
gcaccaacactgaaataaccccaaa

SEQ ID NO: 223 - DNA, 31 bp, Artificial sequence
  Primer
aatgataatccaattgacttcaaattaggac

SEQ ID NO: 224 - DNA, 32 bp, Artificial sequence
  Primer
ttttgttcaacatatttcttttgtttttactc

SEQ ID NO: 225 - DNA, 31 bp, Artificial sequence
  Primer
tattttgattacatgaagttactgatgagcc

SEQ ID NO: 226 - DNA, 30 bp, Artificial sequence
  Primer
tacattttcgtagattagttcaacattaac

SEQ ID NO: 227 - DNA, 23 bp, Artificial sequence
  Primer
tattagaaacggaagctttccag

SEQ ID NO: 228 - DNA, 25 bp, Artificial sequence
  Primer
attgtttttggttatggaggaatcg

SEQ ID NO: 229 - DNA, 29 bp, Artificial sequence
  Primer
tatttacctttattccatggaagattttt

SEQ ID NO: 230 - DNA, 31 bp, Artificial sequence
  Primer
gcagaatcagtttcactaggattttttccca

SEQ ID NO: 231 - DNA, 29 bp, Artificial sequence
  Primer
gaaaatgataatgacattaacaacttcag

SEQ ID NO: 232 - DNA, 31 bp, Artificial sequence
  Primer
attgggataaaagtgaatttgtaattgattg

SEQ ID NO: 233 - DNA, 19 bp, Artificial sequence
  Primer
catcatcttcttccacctc

SEQ ID NO: 234 - DNA, 30 bp, Artificial sequence
  Primer
tactgttattattgttaggttgattggcgg

SEQ ID NO: 235 - DNA, 30 bp, Artificial sequence
  Primer
accaataataatggtagtctttattcaagt

SEQ ID NO: 236 - DNA, 29 bp, Artificial sequence
  Primer
agaaacattttcattctaatgaaaggttc

SEQ ID NO: 237 - DNA, 24 bp, Artificial sequence
  Primer
atactgaagacatcgtcaagaagg

SEQ ID NO: 238 - DNA, 32 bp, Artificial sequence
  Primer
tttaagtaaatcttgaacacaacttcttaaac

SEQ ID NO: 239 - DNA, 20 bp, Artificial sequence
  Primer
tgccaagaatataaccgctg

SEQ ID NO: 240 - DNA, 29 bp, Artificial sequence
  Primer
gagtatatgttttatattccatcagtttt

SEQ ID NO: 241 - DNA, 23 bp, Artificial sequence
  Primer
agcctcatgaaaaagtgatccaa

SEQ ID NO: 242 - DNA, 25 bp, Artificial sequence
  Primer
tatcatcaggtaaatgtgaggtagt

SEQ ID NO: 243 - DNA, 29 bp, Artificial sequence
  Primer
tttagtttcatattcacgacgtatttatc

SEQ ID NO: 244 - DNA, 30 bp, Artificial sequence
  Primer
gatgtttcttcataaacttgaatggttgct

SEQ ID NO: 245 - DNA, 32 bp, Artificial sequence
  Primer
aaatgaaaaattatacggatatgtccaaggag

SEQ ID NO: 246 - DNA, 29 bp, Artificial sequence
  Primer
caataatctttttacagataacgtcattt

SEQ ID NO: 247 - DNA, 23 bp, Artificial sequence
  Primer
ctgaaatttggtgctcaaatcgt

SEQ ID NO: 248 - DNA, 29 bp, Artificial sequence
  Primer
ttacagctaatattgttctctttgtattg

SEQ ID NO: 249 - DNA, 28 bp, Artificial sequence
  Primer
gtcaccatcatctagttacgccctacca

SEQ ID NO: 250 - DNA, 26 bp, Artificial sequence
  Primer
taaacaggagaaatggtgacatttat

SEQ ID NO: 251 - DNA, 31 bp, Artificial sequence
  Primer
agaaaaatttatttatcgtctcgaattaaac

SEQ ID NO: 252 - DNA, 18 bp, Artificial sequence
  Primer
ccaccaacaccaacggat

SEQ ID NO: 253 - DNA, 29 bp, Artificial sequence
  Primer
tgaagcttttttcaaacttttctattact

SEQ ID NO: 254 - DNA, 21 bp, Artificial sequence
  Primer
ttcacttttaggttgctgtgg

SEQ ID NO: 255 - DNA, 31 bp, Artificial sequence
  Primer
ttcaatcacatcattacaatgttaaaacacg

SEQ ID NO: 256 - DNA, 29 bp, Artificial sequence
  Primer
tattaacaatattattaacattggtagga

SEQ ID NO: 257 - DNA, 18 bp, Artificial sequence
  Primer
gcaacattggaataccat

SEQ ID NO: 258 - DNA, 18 bp, Artificial sequence
  Primer
ctgccgctgctgcagccg

SEQ ID NO: 259 - DNA, 24 bp, Artificial sequence
  Primer
tgacttgagtgatttagcaagtga

SEQ ID NO: 260 - DNA, 22 bp, Artificial sequence
  Primer
gttggtcactttgaaaatacga

SEQ ID NO: 261 - DNA, 27 bp, Artificial sequence
  Primer
taatgctaatatattttttgtgatact

SEQ ID NO: 262 - DNA, 20 bp, Artificial sequence
  Primer
gaaaaaagcttcagcaaagt

SEQ ID NO: 263 - DNA, 28 bp, Artificial sequence
  Primer
tctaatatttgtgtttatatatcatcat

SEQ ID NO: 264 - DNA, 1213 bp, Tetranychus urticae
  misc_feature (780)..(780): n is a, c, g, or t
ggcacgaggcatcacctcatttccagcctggtccaacacctccaagagacgagaaaccaacattggaggaaatatctcgtgtaaatggtaaaaataaaaaaaactggaaacctcgtacaatttattcaagtattcaacttcaacagcttaacagacgatttcaaaggacacagtatttagctttacccgaaagagctgagttagcagcatcactaggattgactcaaacacaggttaaaatatggctacaaaataaacgttcaaaaaacaagaaaatgcaaaaagctcaggaagctgtgaatggaggtggacaagtgaatggttcacaaggtgttgcctgtggtactggtggtggtcgacgaggtcgaggaggtaatcaaggtcaagggcagaatcaatcgcagcaacagcaacaagcacaacaacaggcccaacatcagcagcagcaacagcagcagcaacaacaccaacaacaacaacaccaacagcaccaacaacaacagcaacaagctcaagtccaacaaactctttcaaatcaacagcaatcacttgatactcaaccaggagttggtctatcctcgggttcacctttcatcaaaggtgaaggttacattcctcaacattcccctgaggtaccaagtgaaagccatacaccattacattcatcacttggtccaaatccgggttccaatggtgtcaactcaaatggccctggtgcacctgttaatagtatcggtaatgcaacctaggaaactcccttgtatcctcgatagtctcctnttcatcttnttcttcctcttcctcaacagcctctggtcctgttcttactcgttccccggttcttccggtttactctgaatcaccacttaactcatcacaaacgattgctcgaagttcacctggaaccattcattcaccttcccacactcccaaccaatggtcctcaatgggtcagataacaccaaaacaagagatcggtcttggcctgggttcctcatcgtcctcctcatcgtcaacctcttcctcttctgtgggtggaggcagtgcactaggtaacactggacatcctccttatccacctccacatccatcaatgtacagcaatagttacgtcagcccttacagttggtaccatcatgaccctcaatccatcaaccctcaactttattaacttcaccaacataaatcgccgataaaaaaaaaaaaaaaaaac

SEQ ID NO: 265 - DNA, 37 bp, Artificial Sequence
  Primer
catctagaggcgcgccatcagcagcagcaacagcagc

SEQ ID NO: 266 - DNA, 39 bp, Artificial Sequence
  Primer
ttggatccatttaaatcccaggccaagaccgatctcttg

SEQ ID NO: 267 - DNA, 2800 bp, Artificial Sequence
  plasmid pB-AGRIKOLA-Tetur17g02200
ggccgctcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgaccctctggtctaccccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagcaagtttgtacaaaaaagcaggcttaatgcaaaaagctcaggaagctgtgaatggaggtggacaagtgaatggttcacaaggtgttgcctgtggtactggtggtggtcgacgaggtcgaggaggtaatcaaggtcaagggcagaatcaatcgcagcaacagcaacaagcacaacaacaggcccaacatcagcagcagcaacagcagcagcaacaacaccaacaacaacaacaccaacagcaccaacaacaacagcaacaagctcaagtccaacaaactctttcaaatcaacagcaatcacttgatactcaaccaggagttggtctatcctcgggttcacctttcatcaaaggtgaaggttacattcctcaacattcccctgaggtaccaagtgaaagccatacaccattacattcatcacttggtccaaatccgggttccaatggtgtcaactcaaatggccctggtgcacctgttaatagtatcggtaatgcaaacctaggaaactcccttgtatcctcgatagtctcctcttcatcttcttcttcctcttcctcaacagcctctggtcctgttcttactcgttccccggttcttccggtttactctgaatcaccacttaactcatcacaaacgattgctcgaagttcacctggaaccattcattcaccttcccacactcccaaccaatggtcctcaatgggtcagataacaccaaaacaagagatcggtcttggcctgggttcctcatcgtcctcctcatcgtcaacctcttcctcttctgtgggtggaggcagtgcactaggtaacactggacatcctccttatccacctccacatccatcaatgtacagcaatagttacgtcagcccttacagttggtaccatcatgaccctcaatccatcaaccctcaactttattaatacccagctttcttgtacaaagtgggtggtctcgaggaattcggtaccccagcttggtaaggaaataattattttcttttttccttttagtataaaatagttaagtgatgttaattagtatgattataataatatagttgttataattgtgaaaaaataatttataaatatattgtttacataaacaacatagtaatgtaaaaaaatatgacaagtgatgtgtaagacgaagaagataaaagttgagagtaagtatattatttttaatgaatttgatcgaacatgtaagatgatatactagcattaatatttgttttaatcataatagtaattctagctggtttgatgaattaaatatcaatgataaaatactatagtaaaaataagaataaataaattaaaataatatttttttatgattaatagtttattatataattaaatatctataccattactaaatattttagtttaaaagttaataaatattttgttagaaattccaatctgcttgtaatt

SEQ ID NO: 268 - protein, 248 aa, Artificial
  Amino acid sequence encoded by plasmid pB-AGRIKOLA-Tetur17g02200
HEASPHFQPGPTPPRDEKPTLEEISRVNGKNKKNWKPRTIYSSIQLQQLNRRFQRTQYLALPERAELAASLGLTQTQVKIWLQNKRSKNKKMQKAQEAVNGGGQVNGSQGVACGTGGGRRGRGGNQGQGQNQSQQQQQAQQQAQHQQQQQQQQQHQQQQHQQHQQQQQQAQVQQTLSNQQQSLDTQPGVGLSSGSPFIKGEGYIPQHSPEVPSESHTPLHSSLGPNPGSNGVNSNGPGAPVNSIGNAT

(19) United States
(12) Patent Application Publication
(10) Pub. No.: US 2012/0331582 A1
(43) Pub. Date: Dec. 27, 2012

(54) METHOD TO CONTROL SPIDER MITES

(75) Inventors: Miodrag Grbic, London (CA); Vojislava Grbic, London (CA); Pierre Hilson, Brussel (BE); Stephane Rombauts, Gent (BE); Yves Van De Peer, Gent (BE)

(73) Assignees: The University of Western Ontario, London, ON (CA); VIB VZW, Leuven (BE)

(21) Appl. No.: 13/501,240

(22) PCT Filed: Oct. 13, 2010
(86) PCT No.: PCT/EP2010/065311

§ 371 Date: Jul. 25, 2012

(30) Foreign Application Priority Data
Oct. 14, 2009 (EP) 09173040.8

(51) Int. Cl. A01H 5/00; A01H 1/00

(52) U.S. Cl. 800/279; 800/302

(57) ABSTRACT

The present invention relates to a method of controlling spider mites on plants. More specifically, the invention relates to plants expressing RNAi of one or more essential genes of the spider mite, and the use of those plants to control the spider mite proliferation into pest proportions. In a preferred embodiment, the spider mite is Tetranychus urticae.

### CROSS-REFERENCE TO RELATED APPLICATIONS

This is a national phase entry under 35 U.S.C. §371 of international Patent Application PCT/EP2010/065311 filed on Oct. 13, 2010, published in English as International Patent Publication No. WO 2011/045333, which claims the benefit under Article 8 of the Patent Cooperation Treaty to European Patent Application Serial No. 09173040.8, filed Oct. 14, 2009.

### TECHNICAL FIELD

The present invention relates to a method to control spider mites on plants. More specifically, the invention relates to plants, expressing RNAi of one or more essential genes of the spider mite, and the use of those plants to control the spider mite proliferation into pest proportions. In a preferred embodiment, the spider mite is Tetranychus urticae.

### BACKGROUND

Spider mites are arthropods, belonging to the subphylum of chelicerates (scorpions, horseshoe crabs, spiders, mites and ticks). The mites include different species that can be parasitic on vertebrate and invertebrate hosts, predators, or plant feeding. Within the mites, the spider mites group the web-spinning species that feed on plants.

Spider mites, and particularly T. urticae (two-spotted spider mite) is one of the major pests in agriculture. It is extremely polyphagous and feed on over 1000 plant species. Moreover, it shows a rapid development (generation time of seven days in a hot season). T. urticae represent a key pest for greenhouse crops, annual field crops and many horticultural crops, such as peppers, tomatoes, potatoes, beans, corn, strawberries and roses. It is widespread all over the world, and occurs freely in nature in regions with a warm and dry climate.

Spider mites cause yellow flecks on the leaf surface, and upon heavy infestation, leaves become pale, brittle and covered in webbing. This damage can cause severe reduction in yield.

Spider mites are particularly important pests for vegetables. Spider mites cause significant damage to greenhouse tomato, cucumber and pepper crops.

Given the short generation time and high reproduction rate of spider mites, it is expected that spider mites, with the climate change, will become one of the major pests for crops as well. Devastating effects of spider mites are already creating enormous problems for the agricultural production in Southern Europe.

Spider mite control, currently, is mainly done by specific miticides, as normal insecticides have normally little effect on mites. Miticides have been disclosed, amongst others, in WO03014048 and in WO2007000098. However, miticides are polluting chemicals, and the application may not always be efficient, as spider mites are often protected by a web under the leaves.

Recently, the RNA interference (RNAi) technology was developed as an attractive alternative in the control of insect pests (Gordon and Waterhouse, 2007; Baum et al., 2007; Mao et al., 2007). RNAi is based on sequence-specific gene silencing that is triggered by the presence of double-stranded RNA (dsRNA). RNAi can be used in plants, animals and insects, but the mechanism depends upon endogenous enzymes present and the efficacy depends upon the host organism used (Gordon and Waterhouse, 2007). Khila and Grbic (2007) demonstrated that dsRNA and short interfering RNA (siRNA) can be used for gene silencing in T. urticae, by using a maternal injection protocol to deliver interfering RNAs into the maternal abdomen. This methodology has been used to silence Distal-less, a conserved gene involved in appendage specification in metazoans.

However, gene silencing has never been used in pest control for spider mites. One reason is the uncertainty whether RNAi, supplied in the food, would be functional. Another reason is the lack of sequence data of spider mites, making a selection of mite-specific genes that are lethal when knocked out by RNAi impossible.

### DISCLOSURE

We sequenced and annotated the genome of T. urticae. This effort allowed us to pinpoint a set of essential mite-specific genes without relevant plant or mammalian orthologs. From these sequences, RNAi loops were designed that were specific for one essential mite gene, without interfering with the expression in plants or in mammals. Surprisingly, we found that expressing RNAi in a plant derived from those genes, is sufficient to interfere with the spider mite's development and physiology that is feeding on this plant, resulting in death as a consequence.

A first aspect of the invention is a transgenic plant expressing RNAi derived from a spider mite. Preferably, RNAi is derived from an essential gene of the spider mite. Even more preferably, the RNAi is derived from a gene-specific region (GSR) of the essential genes. A “transgenic plant” can be any plant that is, as wild-type, sensitive to spider mite infection, including, but not limited to, members of the citrus family (lemon, oranges, . . . ), grapefruit, different varieties of Vitis, corn, as well as Solanaceae like tomatoes, cucumber, . . . and ornamental flowers. “Derived” as used here, means that the gene region that is transcribed (including the non-coding regions) is used to design the RNAi; preferably, the RNAi comprises an antisense fragment of the transcribed region. Even more preferably, it consists of an antisense region of the transcribed region. The RNAi comprises only a part of the transcribed mRNA. A “GSR” is a gene region without homology with other mite genes and without homology with the host genome, as determined according to Example 1. A GSR allows the design of RNAi that is specific for the target gene, without interfering with other mite genes or with plant or mammalian genes. An “essential gene” as used here means that the inactivation of the gene is blocking growth and/or development of the mite and may result in the death of the mite. Preferably, the essential gene is selected from the group consisting of GABA receptor gene, stem cell gene, neutralized gene, HOX gene, DEV gene, Cytochrome C gene, Hedgehog gene, NADH dehydrogenase gene, Ryanoid receptor gene, sodium channel gene, acetylcholine esterase gene, son of sevenless gene, prospero gene, acetyl choline receptor gene and distal-less gene (Dll). Preferably, the spider mite is T. urticae. In one preferred embodiment, the RNAi is derived from the T. urticae distal-less gene (RNAi indicated as Tetur17g02200-SEQ ID NO:86); preferably, it is comprising the sequence between the primers as shown in FIG. 1. In another preferred embodiment, the RNAi is comprising a sequence selected from the group consisting of SEQ ID NOS:1-87. Even more preferred, the RNAi is comprising a sequence; even more preferably, consisting of a sequence selected from the group consisting of SEQ ID NOS:1, 2, 4, 6, 9, 14, 18, 20, 21, 22, 24, 33, 34, 35, 36, 37, 38, 39, 46, 49, 50, 63, 75, 86 and 87. Most preferably, the RNAi is comprising a sequence; even more preferably, consisting of a sequence selected from the group consisting of SEQ ID NOS:2, 18, 22, 75 and 86.

Although, preferably, the inactivation of the mites is obtained by expressing a single RNAi species, it is clear for the person skilled in the art that the same effect may be obtained by expressing more than one RNAi species, in order to obtain a stronger inhibition.

Another aspect of the invention is a method to improve mite resistance in plants, comprising the expression of RNAi derived from spider mite. Preferably; the RNAi is derived from an essential gene from spider mite; even more preferably, the RNAi is derived from a gene-specific region (GSR) of the essential gene. Preferably, the essential gene is selected from the group consisting of GABA receptor gene, stem cell gene, neutralized gene, HOX gene, DEV gene, Cytochrome C gene, Hedgehog gene, NADH dehydrogenase gene, Ryanoid receptor gene, sodium channel gene, acetylcholine esterase gene, son of sevenless gene, prospero gene, acetyl choline receptor and distal-less gene (Dll). Preferably, the spider mite is T. urticae. In one preferred embodiment, the RNAi is derived from the T. urticae distal-less gene; preferably it is comprising the sequence between the primers as shown in FIG. 1. In another preferred embodiment, the RNAi is derived from a sequence comprising a sequence selected from the group consisting of SEQ ID NOS:1-87. Even more preferred, the RNAi is comprising a sequence; even more preferably, consisting of a sequence selected from the group consisting of SEQ ID NOS:1, 2, 4, 6, 9, 14, 18, 20, 21, 22, 24, 33, 34, 35, 36, 37, 38, 39, 46, 49, 50, 63, 75, 86 and 87. Most preferably, the RNAi is comprising a sequence; even more preferably, consisting of a sequence selected from the group consisting of SEQ ID NOS:2, 18, 22, 75 and 86.

### BRIEF DESCRIPTION OF THE DRAWINGS

FIG. 1: Sequence of the Tetranychus urticae distal-less gene (Dll) (SEQ ID NO:264) and the primers used (TuDII_ARBF and TuDII_ARBR) (SEQ ID NO:265 and 266, respectively). The primer regions in the distal-less sequence are underlined. The fragment in between the primers is used in the RNAi construct. The amino acid sequence is identified as SEQ ID NO:268.

FIG. 2: Construct used to express TuDll-RNAi transgene in Arabidopsis.

FIG. 3: Arabidopsis plants expressing dsRNA against Tu-D11 suppress mite development. A) Northern blot analysis showing siRNAs against TuDll spider mite gene; Col is a control, not expressing the transgene. B) Effect of plant-produced TuDll-RNAi (Lines 1-5) on spider mite development. Note that number of eggs deposited on transgenic plants is lower than in the Col control. Also, the number of eggs correlates with the amount of TuDll-RNAi expressed.

FIG. 4: Plasmid map of pB-AGRIKOLA-Tetur17g02200.

### DETAILED DESCRIPTION OF THE INVENTION

### Examples

### Example 1

### Growth Inhibition of T. urticae by Feeding on TuDll-RNAi Transgenic Arabidopsis

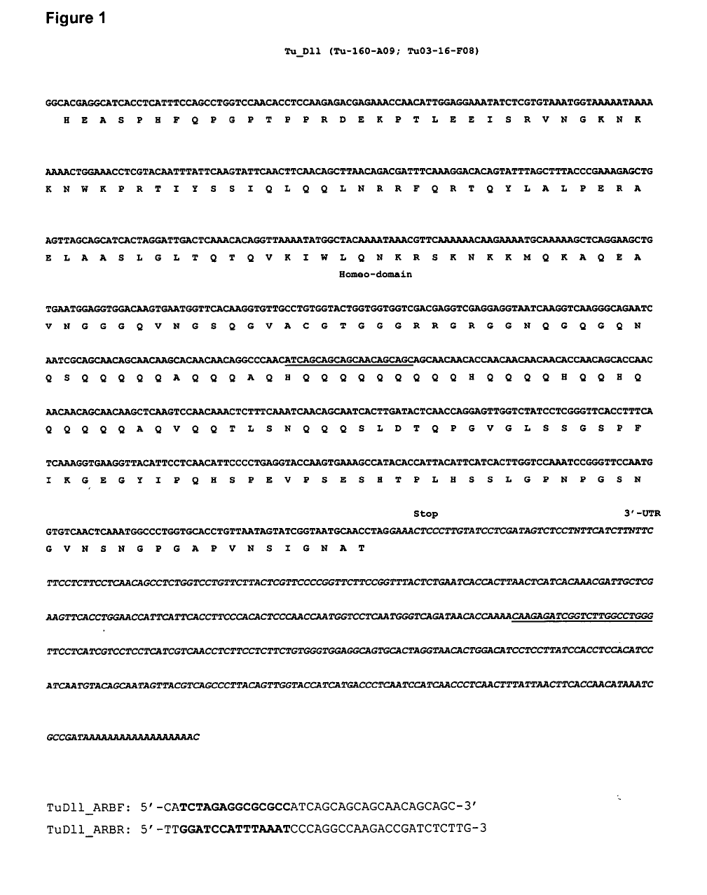

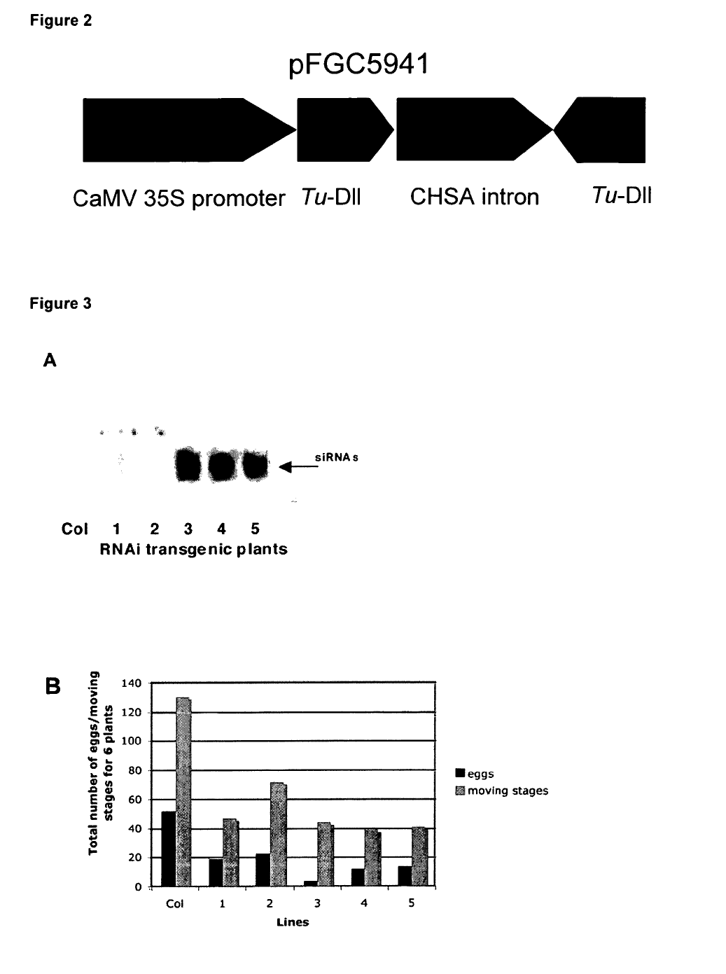

The T. urticae ortholog of the drosophila Dll distal-less gene was identified in the genomic sequence, using the motifs of the distal-less family (Fonseca et al., 2009). Distal-less is a transcription factor that plays an important role in neuronal development (Cobos et al., 2005). An RNAi fragment is designed on the base of its specificity (no significant homology with other T. urticae genes, neither with the Arabidopsis genome). The RNAi fragment, as well as the primers used to isolate it, is shown in FIG. 1. The fragment was amplified, and cloned under control of the CaMV 35S promoter, to result in the Ti-based plasmid pFGC5941 (FIG. 2). The plasmid was transformed using the Agrobacterium-mediated transformation into Arabidopsis thaliana (Col). The expression of the RNAi in different transformed lines was tested by Northern blot (FIG. 3, Panel A). Spider mites were allowed to feed on five transformed lines and a control plant. All transformed plants showed an inhibition of mite development, both of the moving stages and the number of eggs on the plant. A correlation between the expression level of RNAi and the number of eggs on the transgenic plants was found (FIG. 3, Panel B), proving that the expression in plants of RNAi of an essential spider mite gene is indeed an efficient way to control the pest.

### Example 2

### RNAi Design for Other Essential Genes

From a list of candidate Tetranychus urticae target genes, coding sequences (CDS, from start-to-stop codons) were collected from the available predicted genes. For each of those genes, overlapping 21mer sequences were designed covering the whole CDS sequences. This was done by extracting, starting from the first nucleotide of the CDS, sub-sequences of 21 nt, with a sliding window, with steps of one nt. For each CDS from the target genes, n−20 oligos of 21 nt were designed, whereby n is the length of the CDS.

Each of these 21mers was blasted (using BLASTN) against the whole Tetranychus urticae genome. In the case of a perfect match, an e-value of 1e−4 is obtained. To allow some mismatch the threshold was set at 0.01. The threshold was lowered to ensure that no 21mer would hit another region on the genome with a small sequence difference of 1 or 2 nt, thereby ensuring the gene specificity for the RNAi.

Gene-specific regions (GSR), ideally being between 150 and 500 nt, were identified as regions for which, over the whole region, none of the consecutive 21mers derived from this region gave a hit with another sequence from the T. urticae (using the threshold as described above).

The GSR that did meet the above conditions were subsequently blasted (BLASTN, same thresholds) against the Arabidopsis genome. Arabidopsis was chosen, as it is used as host in the proof of principle experiments. This step is to make sure that no Arabidopsis genes could be targeted by the RNAi constructs introduced and that might thus affect Arabidopsis directly; GSR can be blasted against other genomes for optimizing the RNAi in other plant hosts.

All GSR that fulfilled the above criteria (SEQ ID NOS:1-85) were then used as input for primer design. The primers where designed using the OSP perl package, and as a parameter, the melting temperature was set at the 55° C. to 65° C. range in a first run (Table 1). Those targeted GSR that did not succeed in obtaining a primer pair were submitted again to the same design procedure, with slightly more relaxed primer lengths allowed (Table 2). If, with those conditions, still no primers could be designed, melting temperature range was relaxed (50° C. to 70° C.) for a third attempt (Table 3).

TABLE 1
primers designed after 1 run

| SEQ_ID | 5_PRIMER | 3_PRIMER |
|---|---|---|
| 0_197_ | ATAAAATCTCCAAGCATAGTACGAGTT (SEQ ID NO: 88) | TTAACCACAGTCACTCGACCTTCA (SEQ ID NO: 89) |
| Tetur41g00290 | | |
| 0_228_ | No Primers could be designed with these | |
| Tetur30g02230 | criteria | |
| 1066_1216_ | TGATTGAATTCACTTTTTCGCACAT (SEQ ID NO: 90) | AAATAACTGAATCTGGCCAAGTTATTA (SEQ ID NO: 91) |
| Tetur01g13610 | | |
| 1126_1276_ | No Primers could be designed with these | |
| Tetur19g01440 | criteria | |
| 114_520_ | CTAAAAATCTAATTGCAGTGGTAG (SEQ ID NO: 92) | CGTTTATCTGGCAATGGAG (SEQ ID NO: 93) |
| Tetur01g13610 | | |
| 1173_1324_ | AATGTTTTCTTTGTGCAAGTTTCTTATC (SEQ ID NO: 94) | GCTGGAAGAGTAAAATGTTTAGGT (SEQ ID NO: 95 |
| Tetur01g21600 | | |
| 1186_1376_ | ACCTGAGAATCTTTGAGACC (SEQ ID NO: 96) | ATCCTCATCACAACAACCTGAC (SEQ ID NO: 97) |
| Tetur14g00120 | | |
| 1204_1399_ | TAACCTCTTGATCCAGTAAAGCTTCAAT (SEQ ID NO: 98) | GTTTATTAGCTGGTCGTTATGCAC (SEQ ID NO: 99) |
| Tetur09g01840 | | |
| 1224_1532_ | CAAGGAGGTTTCATCAGGATA (SEQ ID NO: 100) | ATGAACATAATTAAAACCTGGTCTTTCG (SEQ ID NO: 101) |
| Tetur31g00990 | | |
| 1236_1391_ | No Primers could be designed with these | |
| Tetur20g01760 | criteria | |
| 1266_1490_ | CTGTCGATTGAACCCTGCAT (SEQ ID NO: 102) | TGTGAACATTGTTCCCATCAACAT (SEQ ID NO: 103) |
| Tetur16g00420 | | |
| 1326_1516_ | No Primers could be designed with these | |
| Teturl9g01440 | criteria | |
| 1506_1673_ | TAAGCATAATAAGTTCTGATAACATCC (SEQ ID NO: 104) | TCTTTGAATGTTGAGTCGGAATG (SEQ ID NO: 105) |
| Tetur01g13610 | | |
| 1564_1794_ | No Primers could be designed with these | |
| Tetur20g01760 | criteria | |
| 161_321_ | CACAAACATAACTTGGCCTAAATCT (SEQ ID NO: 106) | AAGATCATCGTTTAATGGTAATGTTGT (SEQ ID NO: 107) |
| Tetur02g06230 | | |
| 173_391_ | CCACTGTTGGTGTAAGTTGTGAAT (SEQ ID NO: 108) | TTCAATCACTTGTCGATATGAGC (SEQ ID NO: 109) |
| Tetur01g12090 | | |
| 1761_1957_ | TGGATTGTTGATGGTTAGACTC (SEQ ID NO: 110) | GCTGCTGCGGCTGCAACT (SEQ ID NO: 111) |
| Tetur01g13860 | | |
| 1812_1966_ | No Primers could be designed with these | |
| Tetur06g02480 | criteria | |
| 1821_1979_ | TGATTGGCAACAATTACTCGATAT (SEQ ID NO: 112) | TTTAATGTTGCTAAAAGTGGGCCCAAC (SEQ ID NO: 113) |
| Tetur20g01760 | | |
| 185_411_ | TGGGCTACTGATACCGAGTT (SEQ ID NO: 114) | GCCTGACATAGATGGATGGGA (SEQ ID NO: 115) |
| Tetur05g05120 | | |
| 200_356_ | TGAGATGAGTATTTACAGGGG (SEQ ID NO: 116) | TTACGTTCTTCCTCCTATTCTTCA (SEQ ID NO: 117) |
| Tetur01g12340 | | |
| 2025_2185_ | AATTATTGTTGTCACTAATTTCGTGTAC (SEQ ID NO: | CACCATCATCAAAAAGTAAATGATTCC (SEQ ID NO: 119) |
| Tetur23g02710 | 118) | |
| 210_397_ | ATGGTAACCAAGTTTCAGCTAGA (SEQ ID NO: 120) | CAAATCAGGTTAGCTCATACAGACA (SEQ ID NO: 121) |
| Tetur12g05390 | | |
| 2129_2321_ | No Primers could be designed with these | |
| Tetur20g01760 | criteria | |
| 226_459_ | AACATAACCATAAACATCACCACC (SEQ ID NO: 122) | GTGTAACTGTTGGTGATCCAGTTC (SEQ ID NO: 123) |
| Tetur01g21600 | | |
| 2296_2467_ | No Primers could be designed with these | |
| Tetur01g13860 | criteria | |
| 232_580_ | CAACAAATCCATATTCAGTCAAGA (SEQ ID NO: 124) | TTCAGAAGATTCAAGTTACTCATGTC (SEQ ID NO: 125) |
| Tetur13g05360 | | |
| 2353_2823_ | CCTGATTTTTAGTAAGCCCATAAATCC (SEQ ID NO: 126) | CATTTTATAATTATTTGACTGCCTGGGT (SEQ ID NO: 127) |
| Tetur06g02480 | | |
| 2371_2583_ | GATAAATTTGTCCCAATAACATTCGTAA (SEQ ID NO: | AATATGAAGATGATTCATCATACTCTG (SEQ ID NO: 129) |
| Tetur23g02710 | 128) | |
| 2380_2694_ | ATAAGCAGGAGGAGGTTGA (SEQ ID NO: 130) | TTAAACGAAAAAGAAGTCGAACTGG (SEQ ID NO: 131) |
| Tetur16g00420 | | |
| 409_2604_ | CAGTTCAAAGTCACAATTCTCTTTACC (SEQ ID NO: 132) | CAACTACTTGAATCGTTAAGAATTTTCC (SEQ ID NO: 133) |
| Tetur19g01440 | | |
| 246_442_ | No Primers could be designed with these | |
| Tetur01g08220 | criteria | |
| 2581_2750_ | No Primers could be designed with these | |
| Tetur01g13860 | criteria | |
| 2582_2766_ | No Primers could be designed with these | |
| Tetur20g01760 | criteria | |
| 259_421_ | No Primers could be designed with these | |
| Tetur07g08130 | criteria | |
| 2651_2803_ | CAACGATTTCTCTCTCCAACCA (SEQ ID NO: 134) | TGCCAGGCAATTGACTTTGTACGA (SEQ ID NO: 135) |
| Tetur19g01440 | | |
| 2685_2839_ | TGTTTGACTGCCGATGAGA (SEQ ID NO: 136) | TTGTTGAATGAAGAAGACGACCTTT (SEQ ID NO: 137) |
| Tetur19g0154 | | |
| 2753_2877_ | ATGAATGCTTTTGCCAACGG (SEQ ID NO: 138) | GTTAATATTTGTTCTAGCTCTAACTAG (SEQ ID NO: 139) |
| Tetur06g02480 | | |
| 2809_2985_ | AATCAATTTTTTATGCTTAGGATGGAG (SEQ ID NO: 140) | GAGAAATCGTTGAAACGGTCAACTT (SEQ ID NO: 141) |
| Tetur19g01440 | | |
| 281_523_ | TAATGGGCAAAGGAATGGGCGA (SEQ ID NO: 142) | CTTTTCAATCTTTTTGTATATACGACTC (SEQ ID NO: 143) |
| Tetur16g02700 | | |
| 3048_3213_ | TGAAACTAAATTATGATGGTGTCGCTT (SEQ ID NO: 144) | TACATTTTTTCTGGAGCGGTTG (SEQ ID NO: 145) |
| Tetur06g02480 | | |
| 3059_3244_ | CAAGAGAAGCTTTTCTAACAACTA (SEQ ID NO: 146) | GGTACTCATCTCTGCTCACCAA (SEQ ID NO: 147) |
| Tetur20g01760 | | |
| 305_460_ | TTGAACCCAATCCATCTGAATTG (SEQ ID NO: 148) | TGGAGTGGCCTTAATTGGAGT (SEQ ID NO: 149) |
| Tetur16g00420 | | |
| 3221_3403_ | No Primers could be designed with these | |
| Tetur06g02480 | criteria | |
| 329_689_ | AATTTGTCCACATTTTGTCGTAAAG (SEQ ID NO: 150) | CAACAACTTATCACCAATAACAGCA (SEQ ID NO: 151) |
| Tetur01g13860 | | |
| 3380_3547_ | GTTCTAAATTTTTGAAGGCAGCTA (SEQ ID NO: 152) | AAATGATTCTGTTATACCAACAGCAGT (SEQ ID NO: 153) |
| Tetur20g01760 | | |
| 339_590_ | GGTATAGTAATCTCGGGTCCTAA (SEQ ID NO: 154) | CAAACACCAAACAATGACAATCAA (SEQ ID NO: 155) |
| Tetur06g02480 | | |
| 3466_3739_ | TTGTTGTTGTTGGTGAAACAGTTGC (SEQ ID NO: 156) | CATTACCCACATCAACATTTATGG (SEQ ID NO: 157) |
| Tetur19g01440 | | |
| 347_817_ | GAGCATCGGAGGTGTCAA (SEQ ID NO: 158) | GACAAAAAAAGGTTATGTTCGTGG (SEQ ID NO: 159) |
| Tetur18g02240 | | |
| 365_571_ | No Primers could be designed with these | |
| Tetur21g03340 | criteria | |
| 372_523_ | CTGAAGAGTGAAATGCTGATGATCGG (SEQ ID NO: 160) | CATCATCATCACCACAAGTCA (SEQ ID NO: 161) |
| Tetur19g01540 | | |
| 3732_3946_ | CAGAGTCAATTGGTGAACCTT (SEQ ID NO: 162) | CAGGCACAGCAACATCAA (SEQ ID NO: 163) |
| Teturl9g01540 | | |
| 3986_4372_ | No Primers could be designed with these | |
| Tetur19g01540 | criteria | |
| 417_589_ | CCCAACCTTTAACAAAAGAAAGCCTA (SEQ ID NO: 164) | ATGCAACAACAAGCTGCTTCA (SEQ ID NO: 165) |
| Tetur08g00500 | | |
| 418_692_ | TCATAATCATCCTCTTCGCCA (SEQ ID NO: 166) | GCATAAATAATAATCGTGATCCTTTAG (SEQ ID NO: 167) |
| Tetur19g01440 | | |
| 445_650_ | TGTTTCAATGTTGATTCCAATGCACT (SEQ ID NO: 168) | AAAATGTACAAAATGCTAGACCTGA (SEQ ID NO: 169) |
| Tetur31g01810 | | |
| 4484_4770_ | AAAGTCAACAACAAGTTCTACATAAGAT (SEQ ID NO: | TCTTTACAAGGAAACTCGTGATCCTG (SEQ ID NO: 171) |
| Tetur20901760 | 170) | |
| 463_801_ | AACATCTTTAGCCATTTGACTGGCTG (SEQ ID NO: 172) | CCACGATTACAGATGGACCTGA (SEQ ID NO: 173) |
| Tetur04g03690 | | |
| 4678_4905_ | TTGAAGAGGAATTGAATTGCCGCAAA (SEQ ID NO: 174) | ATCATCATCAAGCAGCCAC (SEQ ID NO: 175) |
| Tetur19g01540 | | |
| 467_666_ | TTGCCATTCAGCATATTTGACAGGAT (SEQ ID NO: 176) | CTTCACCAAGAATGGCCAC (SEQ ID NO: 177) |
| Tetur10g00660 | | |
| 46_199_ | TTGTTGTGGTTGTCGTTATAACCT (SEQ ID NO: 178) | GCGATTTAACCACACTTTTCCT (SEQ ID NO: 179) |
| Tetur14g00860 | | |
| 4755_5024_ | TCCTCTTCATCGTCACCGAAACA (SEQ ID NO: 180) | ACCACAACCATCACATTGAAC (SEQ ID NO: 181) |
| Tetur01g13860 | | |
| 47_255_ | AAGGTAAGAGTTGAAAACAAATCCAAG (SEQ ID NO: 182) | AGATGATGCAGAAAGACAAACTCAG (SEQ ID NO: 183) |
| Tetur26g02710 | | |
| *494_599_ | TACTCCACTAGAGTTATATCATGAGTCT (SEQ ID NO: | AATGGACGATGAACTGGTTAAATT (SEQ ID NO: 185) |
| Tetur01g08060 | 184) | |
| 50_206_ | No Primers could be designed with these | |
| Tetur01g21600 | criteria | |
| 518_697_ | ACCAATAAACATTTCCTTGTGGTG (SEQ ID NO: 186) | CGAGAAATTTTTGGCTCGTGAT (SEQ ID NO: 187) |
| Tetur01g07940 | | |
| 545_715_ | CAAATTTACACTCTCGAGCGCGAGTT (SEQ ID NO: 188) | TTTGCTGGTTGTTGTTCCTAAAGCAT (SEQ ID NO: 189) |
| Tetur30g02230 | | |
| 5574_6004_ | AAATCATTAATGGTAAGCCTTCAC (SEQ ID NO: 190) | AAACGAGAAAAGGCAACTAAATTGG (SEQ ID NO: 191) |
| Tetur20g01760 | | |
| 566_774_ | No Primers could be designed with these | |
| Tetur07g01500 | criteria | |
| 588_759_ | No Primers could be designed with these | |
| Tetur07g05390 | criteria | |
| 5_168_ | ACAAGTGATTGAATTGAATCGACAAA (SEQ ID NO: 192) | CAATGTGAACCAAAACACCTCT (SEQ ID NO: 193) |
| Tetur01g12090 | | |
| 6075_6322_ | No Primers could be designed with these | |
| Tetur20g01760 | criteria | |
| 643_815_ | TATTTTTTTGCCTCGGGCTGAGGT (SEQ ID NO: 194) | ATCGTTATGATGATGAATTGGGTA (SEQ ID NO: 195) |
| Tetur13g05360 | | |
| 653_806_ | No Primers could be designed with these | |
| Tetur19g01540 | criteria | |
| 694_948_ | TTTACCTTTACGGGGAACCAA (SEQ ID NO: 196) | ATGTGGACAAATTTATGAACGAATCGCT (SEQ ID NO: 197) |
| Tetur01g13860 | | |
| 701_937_ | TCATTCGATTGGTAATGAATCGTATCT (SEQ ID NO: 198) | TGGTTTACCTTGTGATCAACTTAATCT (SEQ ID NO: 199) |
| Tetur21g03340 | | |
| 719_896_ | No Primers could be designed with these | |
| Tetur01g12340 | criteria | |
| *747_1103_ | CGAGTCGAGGTTGACCCACAG (SEQ ID NO: 200) | ATTTTTGTCTCCATTAACTATCGTGTTG (SEQ ID NO: 201) |
| Tetur18g02240 | | |
| 747_966_ | TCTTCTTTGTTGTTTCTTATTGGG (SEQ ID NO: 202) | CAATACAATGAACAAGAAATTGCAGAT (SEQ ID NO: 203) |
| Tetur30g02230 | | |
| 748_1010_ | TAAACTGGAGTGGTTCGCCGTA (SEQ ID NO: 204) | CTCAACAGCAGCAACATGAT (SEQ ID NO: 205) |
| Tetur16g02700 | | |
| 751_910_ | AAATTTTGGTGAATTCATATTCAGACTG (SEQ ID NO: | ATGGAAAAATCTTTGAGGTTAAACATGC (SEQ ID NO: 207) |
| Tetur31g01810 | 206) | |
| 762_1003_ | CACCTTTAACTCCTACTGGAA (SEQ ID NO: 208) | GGTTTAATGGATGACATTTATCAATGG (SEQ ID NO: 209) |
| Tetur07g08130 | | |
| 764_938_ | No Primers could be designed with these | |
| Tetur07g05390 | criteria | |
| 819_1066_ | CTTCCAACACTTGACGAG (SEQ ID NO: 210) | AATAAACATACAAACCGTGAGCC (SEQ ID NO: 211) |
| Tetur06g02480 | | |
| 868_1056_ | No Primers could be designed with these | |
| Tetur14g00860 | criteria | |
| 943_1154_ | TAAAGATCACCGGTTGTCTTGTA (SEQ ID NO: 212) | TTGGTGTTGGTGGCTCGT (SEQ ID NO: 213) |
| Tetur07g05390 | | |
| 944_1108_ | CAAATTCAACATTTTCGGCCATC (SEQ ID NO: 214) | TAAGCCATTAATTAGTGAGAAAGACAT (SEQ ID NO: 215) |
| Tetur19g01440 | | |
| 94_564_ | TACTTGGTGCACTTGTAACAATACGG (SEQ ID NO: 216) | TAACCACAGGCGATATGAG (SEQ ID NO: 217) |
| Tetur01g08060 | | |

TABLE 2
primers designed after two runs

| SEQ_ID | 5_PRIMER | 3_PRIMER |
|---|---|---|
| 0_228_ | ATTTTTGTTTTCAAAGATATCGTGGATACAGG (SEQ ID NO: | AGTGAATTTTGGCTCATCTCAG (SEQ ID NO: 219) |
| Tetur30g02230 | 218) | |
| 1126_1276_ | ATTTTGGTAAAATATACTTGGCAGAAAGA (SEQ ID NO: 220) | AAGTATTTGAAAAATATACCCTTGATATG (SEQ ID |
| Tetur19g01440 | | NO: 221) |
| 1236_1391_ | GCACCAACACTGAAATAACCCCAAA (SEQ ID NO: 222) | AATGATAATCCAATTGACTTCAAATTAGGAC (SEQ ID |
| Tetur20g01760 | | NO: 223) |
| 1326_1516_ | TTTTGTTCAACATATTTCTTTTGTTTTTACTC (SEQ ID NO: | TATTTTGATTACATGAAGTTACTGATGAGCC (SEQ ID |
| Tetur19g01440 | 224) | NO: 225) |
| 1564_1794_ | TACATTTTCGTAGATTAGTTCAACATTAAC (SEQ ID NO: | TATTAGAAACGGAAGCTTTCCAG (SEQ ID NO: 227) |
| Tetur20g01760 | 226) | |
| 1812_1966_ | ATTGTTTTTGGTTATGGAGGAATCG (SEQ ID NO: 228) | TATTTACCTTTATTCCATGGAAGATTTTT (SEQ ID NO: |
| Tetur06g02480 | | 229) |
| 2129_2321_ | GCAGAATCAGTTTCACTAGGATTTTTTCCCA (SEQ ID NO: | GAAAATGATAATGACATTAACAACTTCAG (SEQ ID NO: |
| Tetur20g01760 | 230) | 231) |
| 2296_2467_ | ATTGGGATAAAAGTGAATTTGTAATTGATTG (SEQ ID NO: | CATCATCTTCTTCCACCTC (SEQ ID NO: 233) |
| Tetur01g13860 | 232) | |
| 246_442_ | TACTGTTATTATTGTTAGGTTGATTGGCGG (SEQ ID NO: | ACCAATAATAATGGTAGTCTTTATTCAAGT (SEQ ID NO: |
| Tetur01g08220 | 234) | 235) |
| 2581_2750_ | AGAAACATTTTCATTCTAATGAAAGGTTC (SEQ ID NO: 236) | ATACTGAAGACATCGTCAAGAAGG (SEQ ID NO: 237) |
| Tetur01g13860 | | |
| 2582_2766_ | TTTAAGTAAATCTTGAACACAACTTCTTAAAC (SEQ ID NO: | TGCCAAGAATATAACCGCTG (SEQ ID NO: 239) |
| Tetur20g01760 | 238) | |
| 259_421_ | GAGTATATGTTTTATATTCCATCAGTTTT (SEQ ID NO: 240) | AGCCTCATGAAAAAGTGATCCAA (SEQ ID NO: 241) |
| Tetur07g08130 | | |
| 3221_3403_ | TATCATCAGGTAAATGTGAGGTAGT (SEQ ID NO: 242) | TTTAGTTTCATATTCACGACGTATTTATC (SEQ ID NO: |
| Tetur06g02480 | | 243) |
| 365_571_ | No Primers could be designed with these | |
| Tetur21g03340 | criteria | |
| 3986_4372_ | No Primers could be designed with these | |
| Tetur19g01540 | criteria | |
| 50_206_ | GATGTTTCTTCATAAACTTGAATGGTTGCT (SEQ ID NO: | AAATGAAAAATTATACGGATATGTCCAAGGAG (SEQ ID |
| Tetur01g21600 | 244) | NO: 245) |
| 566_774_ | No Primers could be designed with these | |
| Tetur07g01500 | criteria | |
| 588_759_ | No Primers could be designed with these | |
| Tetur07g05390 | criteria | |
| 6075_6322_ | CAATAATCTTTTTACAGATAACGTCATTT (SEQ ID NO: 246) | CTGAAATTTGGTGCTCAAATCGT (SEQ ID NO: 247) |
| Tetur20g01760 | | |
| 653_806_ | TTACAGCTAATATTGTTCTCTTTGTATTG (SEQ ID NO: 248) | GTCACCATCATCTAGTTACGCCCTACCA (SEQ ID NO: |
| Tetur19g01540 | | 249) |
| 719_896_ | TAAACAGGAGAAATGGTGACATTTAT (SEQ ID NO: 250) | AGAAAAATTTATTTATCGTCTCGAATTAAAC (SEQ ID |
| Tetur01g12340 | | NO: 251) |
| 764_938_ | CCACCAACACCAACGGAT (SEQ ID NO: 252) | TGAAGCTTTTTTCAAACTTTTCTATTACT (SEQ ID NO: |
| Tetur07g05390 | | 253) |
| 868_1056_ | TTCACTTTTAGGTTGCTGTGG (SEQ ID NO: 254) | TTCAATCACATCATTACAATGTTAAAACACG (SEQ ID |
| Tetur14g00860 | | NO: 255) |

TABLE 3
primers designed after 3 runs

| SEQ_ID | 5_PRIMER | 3_PRIMER |
|---|---|---|
| 365_571_ | TATTAACAATATTATTAACATTGGTAGGA (SEQ ID NO: | GCAACATTGGAATACCAT (SEQ ID NO: 257) |
| Tetur21g03340 | 256) | |
| 3986_4372_ | CTGCCGCTGCTGCAGCCG (SEQ ID NO: 258) | TGACTTGAGTGATTTAGCAAGTGA (SEQ ID NO: 259) |
| Tetur19g01540 | | |
| 566_774_ | GTTGGTCACTTTGAAAATACGA (SEQ ID NO: 260) | TAATGCTAATATATTTTTTGTGATACT (SEQ ID NO: 261) |
| Tetur07g01500 | | |
| 588_759_ | GAAAAAAGCTTCAGCAAAGT (SEQ ID NO: 262) | TCTAATATTTGTGTTTATATATCATCAT (SEQ ID NO: 263) |
| Tetur07g05390 | | |

### Example 3

### Expression of RNAi in Plants

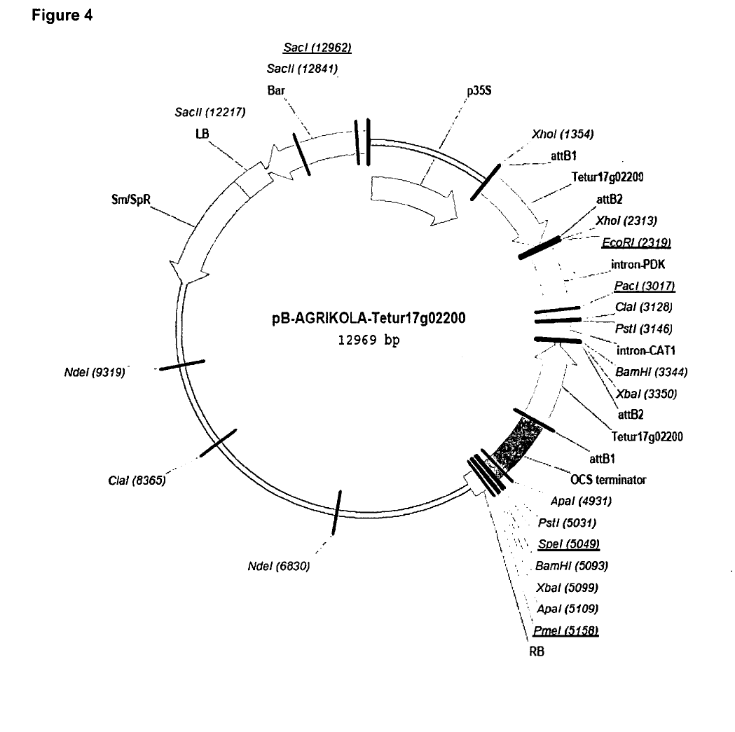

Similar to the RNAi distal-less construct, RNAi constructs of the other essential genes are placed under control of the CaMV 35 S promoter, in pB-Agrikola. The plasmid map of pB Agrikola (carrying the RNAi construct of Tetur17g02200-SEQ ID NO:86) is given in FIG. 4; the sequence of the plasmid is given in SEQ ID NO:267. In a similar way, constructs were made for the RNAi of SEQ ID NOS:2, 18, 22 and 75. The resulting constructs were agro-infiltrated into Arabidopis. RNAi expression is checked by Northern blot. RNAi positive lines are further cultivated to be used in a feeding test.

### Example 4

### Feeding Tests with T. urticae

Arabidopsis plants expressing dsRNA from the selected genes are used in spider mite food tests, and the effect on mite development is measured, as described in Example 1. A reduction in living mites, as well in eggs, on the plants is obtained.

### REFERENCES

Baum J. A., T. Bogaert, W. Clinton, G. R. Heck, P. Feldmann, O. Ilagan, S. Johnson, G. Plaetinck, T. Munyikwa, M. Pleau, T. Vaughn and J. Roberts (2007). Control of coleopteran insect pests through RNA interference. Nature Biotech. 25:1322-1326.
Cobos I., V. Broccoli, and J. L. Rubenstein (2005). The vertebrate ortholog of Aristaless is regulated by Dlx genes in the developing forebrain. J. Comp. Neurol. 483:292-303.
Fonseca N. A., C. P. Vieira, and J. Vieira (2009). Gene classification based on amino acid motifs and residues: the DLX (distal-less) test case. PLoS One, 4:e5748.
Gordon K. H. J and P. M. Waterhouse (2007). RNAi for insect-proof plants. Nature Biotech. 25:1231-1232.
Mao Y. B., W. J. Cai, J. W. Wang, G. J. Hong, X. Y. Tao, L. J. Wang, Y. P. Huang, and X. Y. Chen (2007). Silencing a cotton bollworm P450 monooxygenase gene by plant-mediated RNAi impairs larval tolerance of gossypol. Nat. Biotechnol. 25:1307-1313.

1. A transgenic plant expressing RNAi derived from a spider mite.

2. The transgenic plant according to claim 1, wherein said RNAi is derived from an essential gene of said spider mite.

3. The transgenic plant according to claim 1, wherein said RNAi is derived from the distal-less gene.

4. The transgenic plant according to claim 2, wherein said RNAi is derived from a gene specific region (GSR) from said essential gene.

5. The transgenic plant according to claim 4, wherein said spider mite is Tetranychus urticae.

6. The transgenic plant of claim 5, wherein said RNAi is derived from a GSR selected from the group consisting of SEQ ID NO:1 through SEQ ID NO:87.

7. A method to improve spider mite resistance in a plant, the method comprising:
expressing RNAi derived from spider mite in the plant.

8. The method according to claim 7, wherein said RNAi is derived from an essential gene of said spider mite.

9. The method according to claim 8, wherein said RNAi is derived from the distal-less gene.

10. The method according to claim 8, wherein said RNAi is derived from a a gene specific region (GSR) from said essential gene.

11. The method according to claim 7, wherein said spider mite is Tetranychus urticae.

12. The method according to claim 11, wherein said RNAi is derived from a molecule selected from the group consisting of SEQ ID NO:1 through SEQ ID NO:87.

13. The method according to claim 11, wherein said RNAi is derived from a molecule selected from the group consisting of SEQ ID NO:1, SEQ ID NO:2, SEQ ID NO:4, SEQ ID NO:6, SEQ ID NO:9, SEQ ID NO:14, SEQ ID NO:18, SEQ ID NO:20, SEQ ID NO:21, SEQ ID NO:22, SEQ ID NO:24, SEQ ID NO:33, SEQ ID NO:34, SEQ ID NO:35, SEQ ID NO:36, SEQ ID NO:37, SEQ ID NO:38, SEQ ID NO:39, SEQ ID NO:46, SEQ ID NO:49, SEQ ID NO:50, SEQ ID NO:63, SEQ ID NO:75, and SEQ ID NO:86.

14. The method according to claim 11, wherein said RNAi is derived from a molecule selected from the group consisting of SEQ ID NO:2, SEQ ID NO:18, SEQ ID NO:22, SEQ ID NO:75 and SEQ ID NO:86.

15. A transgenic plant expressing an RNAi derived from a molecule selected from the group consisting of SEQ ID NO:1 through SEQ ID NO:87.

16. The transgenic plant of claim 15, wherein the molecule is selected from the group consisting of SEQ ID NO:1, SEQ ID NO:2, SEQ ID NO:4, SEQ ID NO:6, SEQ ID NO:9, SEQ ID NO:14, SEQ ID NO:18, SEQ ID NO:20, SEQ ID NO:21, SEQ ID NO:22, SEQ ID NO:24, SEQ ID NO:33, SEQ ID NO:34, SEQ ID NO:35, SEQ ID NO:36, SEQ ID NO:37, SEQ ID NO:38, SEQ ID NO:39, SEQ ID NO:46, SEQ ID NO:49, SEQ ID NO:50, SEQ ID NO:63, SEQ ID NO:75, and SEQ ID NO:86.

17. The transgenic plant of claim 16, wherein the molecule is selected from the group consisting of SEQ ID NO:2, SEQ ID NO:18, SEQ ID NO:22, SEQ ID NO:75, and SEQ ID NO:86.

18. The transgenic plant of claim 2, wherein the RNAi is derived from the distal-less gene.

19. The transgenic plant of claim 3, wherein the spider mite is Tetranychus urticae.

20. The transgenic plant of claim 18, wherein the spider mite is Tetranychus urticae.
